SEQUENCE LISTING (32 sequences)

SEQ ID NO: 1 - DNA, 1227 bp, Rattus norvegicus
  CDS (78)..(953)
ggaattcgcaacgcctacacaaagatccctatcactgagcacccggaggcaaggctcagaaccccaggatcagcaacatggagttctcccgtccaccgctagtgcatgtgaagggtatcccactcatcaaatactttgcagagacaattgggccattgcagaacttcacagcctggcctgatgacttgctgatcagcacatacccaaagtctggtactacctggatgagtgagatcctggatatgatctatcagggtggcaagctagagaagtgtggccgcgcccccatctatgcccgggtacccttccttgagttcaaatgtccaggggttccctcaggtcttgaaactttggaagagacaccagccccacggctccttaagacacatctgcccctgtccttgctccctcagagtctgctggatcagaaggtcaaggtgatctacattgcccgaaatgcaaaggatgtggttgtctcctattataacttctacaacatggccaagctgcaccctgatccaggcacctgggacagcttcttggagaacttcatggatggggaagtgtcctatgggtcgtggtaccagcacgtgaaggagtggtgggagctgagacacactcaccctgttctctatctcttctatgaagacataaaggagaaccccaaaagggagatcaagaagattctagagtttttggggcgctctctacccgaggagactgtggattccattgttcaccacacatctttcaagaaaatgaaagagaactgcatgactaactacacaaccatccccactgagattatggaccacaatgtttctcccttcatgaggaaaggtactactggggactggaaaaataccttcactgtagcccagaatgagcgctttgatgcccactatgctaagacaatgacagattgtgacttcaagtttcgttgtgaactatgagtggattatggctatactgggaaccaaggcaaactgacacagcccatcatgatctcaagtaaaatgtgatgtgttcaatctacttgttgtatgcctagaggaaatctgagctaagagaataggattggggatgtggctgaggcagagggttttatcaacgcatgtcaggaaagcaatcagtcccaacacctaaaaagaacctaaagtacaaacatgcaaaaaatagtaagataaactatattttacctgaaagaataaatgccactgggaaatg

SEQ ID NO: 2 - protein, 291 aa, Rattus norvegicus
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 3 - DNA, 1629 bp, Rattus norvegicus
  CDS (14)..(649)
cgggtgtctgagaatgcaaagtgccatgttcctggctgtccagcacgactgcgtacccatggacaagagcgcaggcaacggccccaaggtcgaggagaagcgggagaaaatgaagcggacactcttaaaagattggaagacccgtttgagctatttcttgcaaaactcctctactcctgggaagcccaaaactggcaagaaaagcaaacagcaaacttttatcaagccttctcctgaggaagcgctgctctgggcagaagcatttgatgaactgctggccagcaaatatgggcttgctgcattcagagcgtttttaaagtccgagttctgtgaagaaaatattgaattctggttggcttgcgaagacttcaagaaaaccaaatcaccacagaaactgtcctcaaaagcaaggaaaatatacaccgacttcatcgagaaggaagctcccaaagagataaacatagactttcaaacaaaaactctgattgcccaaaatatccaagaggctacaagtggctgcttcaccacagcgcagaagagggtgtacagcctgatggagaacaactcttatcctcgtttcttggaatcagaattttatcaggacttgtgtaaaaagccacagatcaccacggagccccatgctacatgagacagggagacccccaccgaggacattccattctgtctcccgagagcaaaggctgtaacctgccagaaaaaaaaaacaaactgaccttgaattcagcctgagtgttaggaaaacatcgctcagaactattgattcaatgttgggtagtgaatcaggaagccagcaacctaggagagtctctgtgagaagtttcccttgctgtgtgaagagcagagggagggaacagggttctgaatgggttcgtccctgtcaggaaagcagggtgtgagatgaaagatacaatggaatgttgttggagcatttaaaatcaataggtctgggattatgtggccttagctagctggctgtacacctttccctgaactagtccatgttaccacgtagtggttttagttctagttttaatattttagtactaagtaacattataatgtttactgtgtgcaagggtgttgactgacgttcttaggactacagatcattaatactattgtgtcatgtatcactgaaactgaaaaagcgtgtttgcattgttaaatggtgtgtgtgatagagtgaatgctgtgccgtgctgtagaaccagtgtccgttccgagtgccaaaaactgtcttgcgggcagctaaactctgaagtggtttttgaatacttttgaataaatttattttgctaaataatgtcattgaacatttggaagtccagatgtggttctgttgttcgatttcagtaactggaaacatgtcagatataagagagaatgactttctagtaaagaggtatcagtgtgggttaattgttgtgcctgtccactgagaaatcgacagccctgagaagtgaagaatgattgcttccaggaagatgttctctttaatcctatagacatgatgtagtcttaagaaaatcagatcacattcctaggaccactcattgtgaaagtcaaatgtgttatttaataaaaaaaaaaaaaaaaaaaaaaaaaaaa

SEQ ID NO: 4 - protein, 211 aa, Rattus norvegicus
MQSAMFLAVQHDCVPMDKSAGNGPKVEEKREKMKRTLLKDWKTRLSYFLQNSSTPGKPKTGKKSKQQTFIKPSPEEALLWAEAFDELLASKYGLAAFRAFLKSEFCEENIEFWLACEDFKKTKSPQKLSSKARKIYTDFIEKEAPKEINIDFQTKTLIAQNIQEATSGCFTTAQKRVYSLMENNSYPRFLESEFYQDLCKKPQITTEPHAT

SEQ ID NO: 5 - DNA, 2919 bp, Rattus norvegicus
  CDS (110)..(727)
gggattctgacagcttctttgcagagcagaagcaactgtgctcaaaatagaaaccaccgttgctcccgctgcctttctctcctcgctccgaatcccacagttaaacaagatgtgcaaaggactcgctggtctgccggcttcctgcctgaggagcgcaaaggatatgaaacatcggctgggatttctgctgcagaaatcagattcctgtgaacatagttcttcacacagcaagaaggacaaagtagtaacttgccagagggtgagccaagaagaagtcaagaaatgggctgaatcgctggaaaacctgattaaccatgaatgtggactggcagctttcaaagctttcctgaagtcagaatacagtgaggagaacattgacttctggatcagctgtgaggagtacaagaaaatcaaatcaccttctaaactaagtcccaaggccaagaagatctacaatgagttcatctctgtgcaggcaacaaaagaggtgaacctggattcttgcaccagagaggagacaagccggaacatgttagagcccacgataacctgttttgatgaagcccagaagaagattttcaacctgatggaaaaggattcataccgtcgtttcctcaagtctcgattctatcttgacctgaccaatccttccagctgcggggcagagaagcagaaaggagccaagagttctgcagactgcacttccctagtccctcagtgtgcctaattctcacacagaggcagagaaccgaaatgccaagactctatgctctggaaaacctgaggccaaatattgatctgtattaagcatcaatgctttctccacattgtagcctagtatccatgctgcctgccgtgtgtgagtcacttccatgtaaaactagacttagctgtggtgtgggggttttcattggggtaagacccaacttgttttcaattttcccaagtttccttaaagggtgtcaggtgagcaagtctccaaataatggccctgtaggtctggattttcaaagatggttagtagtccaccccttccaacagtttgacctcaagttagttggtacacttcatgtgatgtcacatgcacatgcaatgtcagccagcagcacttcctacagactttcaacgtttaggtgatatttgtgtatgtgtatgtatatattaaatatatagaacatatgcatatacattctgtatatattatatctatgtattcagtatgtgtgaatgtgcatgaatatatatatatatgcatatatatatttgtatatgttcacacatgcacacacatttcttcaagaaaagatgggaaagatccttggtgctcaaattggagtgtgtacacatgcttgcatgcatgttctgacttctggtccttcatactgtgtctgaacagggcaaactaaattaactgccatgcaggctaagaaaggaacactaacttgtaaaaagtgagttccgtcaagccactgggtcttgatggagcatagtcagggcctttgtgtttaatctcgtcaatggcctccacattgagcactgagctatggggtgagtactccgaaaactctgctgtctgactgacacagagctgcctcgcaccggtgattactactagcacttttaagtccctatgctgttggccatccctcctttgcctgggtgctcagggcatgctgactagcagctgggttggtctggagatggatgtttgagaggaatgaaggaatgcggtccaggccttgttgcctagccctacaaatgactactgcttaggtgagggcagaagaccccgaggttatcttaattctttcctctcttgataaggtgctctcctattcctacccccaaagaacgtcacctgtcagatgagcctctttacttccccgtggttggttgctcacgttaggaaccatggccacctgcaagtcattctaagctacccgcttacatttttcaaaagcatcaccagggggaaaggttatgctgtcccaggaggaagtaagctcttatgagctgtaaacccctttttggttttctgctttgaggtaagagcttgttcatcccatcccaaacaccaaagttagcagagaccaacggaaggagagaggaaagaaaaaaaaaaagaaagagaagagaaaagaggaggacgggatatcagttggtaggactactgtttggagctttttaaggcacgtgaaatacttgaaaatttctcatttgtgttatttatgttgttttgtacaatgtttttattactgtattgttttataaatggaggtacaggatattacccgaattattaatgaatgcccaagaagtaattttcttctcattcctctaaaagtcctgcctttgagagcgcacacagactcgtgcatactgcaggcacagctccagtttaaattccatgcatgagcaccattgtggctgcaaagtgaagacactcatagtgtgaatccaagtctcaccattattgaagaagtcagttttctaacagtactgttgtctctcttgacagtcttgcgtataatatccccagagctcccccatcagtggtcctaactcctttttgtaatacacaagtccacagtcgtgtctgtataactctccggtcttgccctaatattatcatgactttgagattgtacctgtactcccagactgctgtctcacaaataggtctgggaaaagtcattctgaatgagaagtaaattattttatgtaatacatcttgagtgtgtcttcagttgtatttgccatcgctctttggtctggcgagcctgcctgctgctggcccctgcaaaggacgctccttccagatgcatgtctgtttctgccatgtgctttataggcctcagcgctgatgcttccaatgaaacacatagatctgaatgataacaggaaataaatgcttcctac

SEQ ID NO: 6 - protein, 205 aa, Rattus norvegicus
MCKGLAGLPASCLRSAKDMKHRLGFLLQKSDSCEHSSSHSKKDKVVTCQRVSQEEVKKWAESLENLINHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLTNPSSCGAEKQKGAKSSADCTSLVPQCA

SEQ ID NO: 7 - DNA, 4614 bp, Rattus norvegicus
  CDS (109)..(1497)
aattccgtgggcggcgggcggcggagaagctccgagcggggccgggcggcggcagattggagtcgcgacacagccgccgtccgcttcgggctccacgtagaaggaaccatgctggccgtcggctgcaccctgctggtcgccctgctggccgcgcccgcagtcgcgctggtccttgggagctgccgcgcgctggaggtggcaaatggtacggtgacgagcctgccaggggccactgttaccctgatctgccctgggaaggaagcagcaggcaatgctaccattcactgggtgtactcaggctcacagagcagagaatggactaccacgggaaacacactggttctgagggccgtgcaggtcaatgacactgggcactatttgtgcttcctggatgatcatctggttgggactgtgcccttgctggtggatgttcccccagaggagcccaagctctcctgcttccggaagaacccccttgtaaatgccttttgtgagtggcatccaagcagcactccctctccaaccacgaaggctgtgatgtttgcaaagaaaatcaacaccaccaatgggaagagtgacttccaggtgccttgccagtattctcagcagctgaaaagcttctcctgcgaggtggagatcctggagggtgacaaagtgtaccacatagtgtcactgtgcgttgcaaacagtgtcggaagcaggtccagccacaatgtagtatttcagagtttaaaaatggtgcagccggatccacctgccaaccttgtggtatcagccatacctggaagcctcgttggctcaaagtcagttggcaagaccctgagtcctgggacccaagttactacttgttgcaattcgagcttcgataccgacctgtatggtcaaagaacgttcacggtgtggccgctccaggtggcccagcatcaatgtgtcatccatgatgccttgcgaggagtaaagcatgtggtgcaggtccgagggaaggaggagtttgacattggccagtggagcaaatggtccccggaggtcacaggcactccttggctagcagagcccaggaccactccggcagggatcccggggaaccccacacaggtctctgttgaagactatgacaaccacgaggatcagtacggaagttctacagaagcaacgagtgtcctcgccccagtgcaaggatcctcgcctatacccctgcccacattcctggtagctggaggaagcctggcgtttggattgcttctctgtgtcttcatcatcttgagactcaagaagaaatggaagtcacaggctgagaaggaaagcaagacgacttctcccccaccgtatcccttgggaccgctgaagccgaccttcctcctggttcctctcctcaccccatcagggtcccataacagctctgggactgacaacaccggaagccacagctgcctgggtgtcagggacccacagtgccctaatgacaacagcaacagagactacttattccccagataattgtctggagggtacctggcagctggcacgcaagtttctcactgccggccccgtccaccagggctgggggcggggtgggcggggctgcagcttcacgatcccacaggagccttgcaaaggttctgagtgggagaagactggtgtgctgcacgggcttcgaaagaaggggctgtgaggagcacgagccatcatgaagagagcctgtgatgactctgaatagagacgcccgcccatcagctacacacctgatggtggctctcaagctatcctctcaggaagcctctgggaggggcgacaaaggctgccccagttgcctagctctggctcactggcccaagctgccttttagcttgaactcctaaaatccaagcaccttggccattctcttcctaggccaccgaggccgcggggaagcttggttctactttccttctcaacacctggagaagcagctgcccggtggtggtgactaacgtatcagggcctgatggcttatgaggaatgacaattaattcctcataagcagtttttaaatgtgaatagtaatcctaggcactgctgacttgaggttttattttcttcgatctcaggacttcaggagagaagcagagcagaagtagagagaggatgggtgtccattgtccgtgtggtacttgagggggatacagcctggaaaacacgtttcctgtccccctactctcccagaagaggtagggggtggcgcctcttccagggcagagagtataactactttacctggccttgcccatactggtttcaactggacttgagctactaggaaaaatgacattcatgcaaaaagaaaactttaactagcaagaatgcacttccactttggtttctagaggactgttgctcctcttgagacgctggaagaggccgctcactgtaccctggtgtatgagccctcaccccccaccccagggtaagtgcagtaactttagtctaaacaccgagtcaggtaaaaatcgaggaaaaaacaaccctgtttcctgtaacagaaaagcctttggtttcgttttgtattttgattttttttttgtcttaaaaagtgtaaaaatagtctgtccatactctgcttcagggaatgacctgtgaatactccccaggcgtgggcaggaagggtgtctgcttcctgctacacctcactgccacctcggccttccttgctttacattcaactgagttgcctcagctgctttcccctggggcgctgaaaaagccagtgatgttggtggtcaccgagaagaccacagagccacagagtaatgctgtgattgaagcgagttacgcaaccacagcaccccacatttgctgtattatagaactatgctaggagcttgccttttcacaaaataccaccaccacgagacgtggcagagctcggaagctgtcaccttgtgccatctgcttgccagctccaaggggccactgacttaagcagttattttctttgtgggctttgttcatttcagggcctgttgctgtcttagaaaaagctctgtcggttgacaaaaacatcagacaggtagtcatgtttatttattttttttccttctttgctaagtctttgggactcaagggtagtaaaaaatgctgtgaaaagggaaacattagaaacagcgatcttcggggaataggtgactgtgcccacgcactgttcttcagtccctcacgtggctctgcccgagtgctgttccaagccaggcagagcaggctggcggaagattgaaatccagatagctcgttatctctgagagctaaatagctttgatctccaagctgttattgctttcactattgtaacaggatagcctccccccccatgtcaaaaggatgcttttcccttttgactttttataagctaagtcagtgaagtctgtttcatctgagctccagcttcgttcagttcgcacaggtgtatgccctcagctgcttcgggcctcagatctgtgctagttgaatggttgtcccatccttgggtcatccttaccagagtttctgcagcccacaggtctgccttgtcaacagtaccacttaacaccagcattcagtgcccaggcagccagatgtggagggtttacccagagatgatttaaacatgaccttaaacgtgtatggtagaacgaggggaacccataccagctcaggttctaaagagatctttgattcttctggcattagtgaaatagctttaaactatttcaaggaagaagccttggccacacccacgacatttggtgacaatcctttctctccatgagccttgtctttacaccttctcacctggctgaaagctcacactgaatctttcctatgtccctggtgtcttgggagaaaggaaactggtatgggcttcactgctggaattggcttggagccagcgtgtggcgcagccctggcagggtgggccaggcttagttatggtgtgctggtttaaggaatgcctggcttgcctggttgcttgggttctgagctgcagagtttcctagcagttctttatggctgacctagttggggaagattcccacactcaactgcaggtggaggtggtgagaaagctgttttcatttggagaggcaggatcagcccaagaagctttcagtgggagagcctacagtgaggctgtacctcactgtgggaggaggcaggccagctggctcaggtcctgggactggcactggggagggtctgccaaaggtccctccagcctgtagtcctagcatagtcgggtgccagttccaggaagtttctatggcaaccttagtgctcattaaggaacattgtcagttttgtgaacatatgctcagatggagatcttgttttcagagaaaggactggtacagtgtgtaacaagctggagcagacagagagactttttggcaagagatcacatccgttaagcagaatacctcagtgctacatgtttttgtctttgagacaatgtttttaaggtttttatgctctgttacctgtaagctgatacctaaaactttctgcaaagtcagggtttttcaatgccttttttttttttttgccattgtttgctttaaagtgaagattgtaactgtttgaaataaataatttctaaaactg

SEQ ID NO: 8 - protein, 462 aa, Rattus norvegicus
MLAVGCTLLVALLAAPAVALVLGSCRALEVANGTVTSLPGATVTLICPGKEAAGNATIHWVYSGSQSREWTTTGNTLVLRAVQVNDTGHYLCFLDDHLVGTVPLLVDVPPEEPKLSCFRKNPLVNAFCEWHPSSTPSPTTKAVMFAKKINTTNGKSDFQVPCQYSQQLKSFSCEVEILEGDKVYHIVSLCVANSVGSRSSHNVVFQSLKMVQPDPPANLVVSAIPGSLVGSKSVGKTLSPGTQVTTCCNSSFDTDLYGQRTFTVWPLQVAQHQCVIHDALRGVKHVVQVRGKEEFDIGQWSKWSPEVTGTPWLAEPRTTPAGIPGNPTQVSVEDYDNHEDQYGSSTEATSVLAPVQGSSPIPLPTFLVAGGSLAFGLLLCVFIILRLKKKWKSQAEKESKTTSPPPYPLGPLKPTFLLVPLLTPSGSHNSSGTDNTGSHSCLGVRDPQCPNDNSNRDYLFPR

SEQ ID NO: 9 - DNA, 2435 bp, Rattus norvegicus
  CDS (66)..(1358)
actgttggctaagccggtctcactgctcctttagtctctttggacactttccaaatatcggggcgatgaccgtcaaaaccgaggctgctcgaagtaccctcacctactccagaatgaggggaatggtagcaatcctcatcgctttcatgaaacagagaaggatgggcctgaacgattttattcagaagcttgccaacaactcctatgcatgcaaacaccctgaagttcaatcctatttgaaaatctcccaacctcaggagcccgaacttatgaacgccaacccctcacctcctccaagtccctctcaacaaatcaacctgggtccatcctcaaatccccacgccaaaccctctgacttccacttcttgaaagtgatcggaaaaggcagttttggaaaggttcttctagcaaggcacaaggcagaagaagcattctatgccgtcaaagttttgcagaagaaagccatcttgaagaagaaggaggagaagcatattatgtcagagcgcaatgttctgttgaagaatgtgaagcaccctttcctggtgggccttcacttctctttccagactgctgacaaactctacttcgtcctagactacattaatggcggagagctgttctaccatctccagagggagcgctgcttcctggaaccccgtgctcgcttctacgcagctgaaatagccagtgccttgggttatctgcactccctaaacatcgtttatcgagacttaaaaccagagaatattctcctagactcacagggacacatcgtcctcactgactttgggctctgcaaggagaacatcgagcacaatgggacaacgtccaccttctgtggcacgcctgagtatctcgctcctgaggttctccataagcagccgtacgaccggacagtggactggtggtgcctcggggctgtcttgtatgagatgctctatggcctgcctccgttctacagccggaacacagccgagatgtatgacaatattctgaacaagcctctccagctgaaaaatatcaccaactcagcaaggcacctgctggagggcctcctgcagaaggaccggaccaagaggctgggtgccaaggatgactttatggagattaagagtcatattttcttctctttgattaactgggatgatctcattaataagaagatcacgcccccatttaacccaaatgtgagcgggcccagtgaccttcggcactttgatcccgagtttactgaggagccggtccccagctccatcgggcgatcccctgacagcatccttgtcacagccagtgtgaaagaagccgcggaagccttccttggcttctcctatgcccctcctatggactccttcctctgaaagctcccaggatggttccgaaggatttcctcagcgtttttctaaagtgttttagttagcctttggtggagttaccagctgacagaacatcttagaagagaaatttgcacaccaggaagcttggcagtcccgcctgccggggcctgcacgcggcttgttgacgcggaagctttccggaagctttccgaagagcacatcctcctctcagtgagctagtgaggtcttcatttcttttcttccttccaacgtggtgctagctctaaaggagcttgagagtgccgcctgagacgcaccttggtctcagtgagaaggaagatgcaggtctaagagggatctccgcaggtctgagctgtgatcaagaatattctgcaatgtgccttttctgagattgtgttagctccaaagcttttcctatcgcagagtgtccagtttttgtttgtttttttttttttgttttgttttttgtttcttttttttcccaacccttgcgtatttcccatgtgtgcagttagtgtgagtgctatgcctgatcacagacagttttgttgtaagcatcaatgtgacacttgcaggacactacaatgtgggacattgtttgtttcttccacatttggaagataaatttatgcgcagactgtttttttttgtaagatataataactaaaacctattgaaacggtcttgcagtgacgagcatccagatgcttgagggaagcattgctgctacaaatatttctatttttagaaagggtttttatggaccaatgccccagttgtcagtcagagccgttggtgttcatgttaaaatgtcacctgcaaaatgggcattatttatggttccccccaacctttgttcattttcttttgcattcctgattattgtgtgtaaagaaagtctgtacattgggttataacactagatatttaaacttacaggcttatttgtaaaccatcattttaatgtcctgtaattaacatggttataacatgtacactcccccctactcaccacacaactttttttgtgtgcggtgaaaccaattttggtttgcaataaaatcttgaaaactatttgcg

SEQ ID NO: 10 - protein, 430 aa, Rattus norvegicus
MTVKTEAARSTLTYSRMRGMVAILIAFMKQRRMGLNDFIQKLANNSYACKHPEVQSYLKISQPQEPELMNANPSPPPSPSQQINLGPSSNPHAKPSDFHFLKVIGKGSFGKVLLARHKAEEAFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNGTTSTFCGTPEYLAPEVLHKQPYDRTVDWWCLGAVLYEMLYGLPPFYSRNTAEMYDNILNKPLQLKNITNSARHLLEGLLQKDRTKRLGAKDDFMEIKSHIFFSLINWDDLINKKITPPFNPNVSGPSDLRHFDPEFTEEPVPSSIGRSPDSILVTASVKEAAEAFLGFSYAPPMDSFL

SEQ ID NO: 11 - DNA, 3862 bp, Rattus norvegicus
  CDS (541)..(3309)
cgactcaggcgtttgactcccagccttctggcacgcccaacccacccggtcttccataagggtcagagaaccaaagagacaacccggacctcctctgggcgccagctcctcggctccaacccgtccagaatcaagcgggattttttttttctttccctctagaaattggctttggtgtgtcgcccgccctctcccctcctcctcgcaactcctcccctctttttttcctccttcttcttcttcctgagacatggcccgggcagtggctcctggaagaggaacaagtgtgggaaaagggagaggaaatcggagctaaatgacaggatgcaggcgacttgagacacaaaaagagacgcgcttctcgcgaattcaggcattgcctcgccgctagccttccccgccaagaccggctgaggattttatggttcttaagcggacttaagagcgttgtttcggattgttaagattcccgtttgctgggttttcctccctcaatcgtgctctcccgcggctgcctggggactggctcggcgaaggaggatggagagggggctgccgttgctgtgcgccacgctcgcccttgccctcgccctggcgggcgctttccgcagcgataaatgtggcgggactataaaaattgaaaacccggggtaccttacatctcccggctaccctcattcttaccatccaagtgagaaatgtgaatggctaatccaagctccggagccctaccagagaatcatgatcaacttcaacccacatttcgatttggaggacagagactgcaagtatgactatgtggaagtgatcgatggagagaatgaaggtggccgcctgtgggggaagttctgtgggaagatcgcaccttcacctgtggtgtcttcagggccatttctcttcatcaaatttgtctctgactatgagacccacggggcaggattttccatccgctatgaaatcttcaagagagggcccgaatgttctcagaactatacagcacctactggagtgataaagtcccctgggttccctgaaaaataccccaacagcttggagtgcacctacatcatctttgcaccaaagatgtctgagataatcctagagtttgaaagttttgacctggagcaagactcaaatcctcccggaggagtgttctgtcgctatgaccggctggagatctgggatggattccctgaagttggccctcacattgggcgttactgtgggcagaaaactcctggccggatccgctcctcttcaggcattctatccatggtcttctacactgacagcgcaatagcaaaggaaggtttctcagccaactacagcgtgctgcagagcagcatctctgaagatttcaagtgtatggaggctctgggcatggaatctggagagatccattctgaccagatcactgcatcttcccagtatggtaccaactggtctgttgagcgctcccgcctgaactaccctgaaaacgggtggacaccaggagaggactcctacagggagtggatccaggtggacttgggcctcctgcgattcgttactgctgtggggacacagggtgccatttccaaggaaaccaagaagaaatattatgtcaagacttacagagtagacatcagctccaacggagaggactggatcaccctgaaggagggaaataaagccattatctttcagggaaacaccaatcccacggatgttgtctttggagttttccccaaaccactgataactcgatttgtccgaatcaaacctgcatcctgggaaactggaatatctatgagatttgaagtttatggctgcaagataacagattacccttgctctggaatgttgggcatggtgtctggacttatttcagactcccagattacagcatccaaccaaggagacaggaactggatgccagaaaacatccgcctggtgaccagtcgaaccggctgggccctgccaccctcaccccacccatacatcaatgaatggctccaagtggacctgggagatgagaagatagtaagaggtgtcatcattcaaggtgggaagcaccgagaaaacaaagtgttcatgaggaagttcaagatcgcctacagtaacaatggttctgactggaaaatgatcatggatgacagcaagcgcaaggctaagtcttttgaaggcaacaacaactatgacacacctgagctccgggcctttacacctctctccacaagattcatcaggatctaccccgagagagccacacatagtgggctcggactgaggatggagctactgggctgtgaagtagaagtgcctacagctggacccacgacacccaatgggaaccccgtggacgagtgtgacgatgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggaacatgacagccacgcgcagctcaggtggagggtgctgaccagcaagacggggcccattcaggaccacacaggagatggcaacttcatctattcccaagctgatgaaaatcagaaaggcaaagtagcccgcctggtgagccctgtggtctattcccagagttctgcccactgcatgaccttctggtatcacatgtccggctctcatgtgggtacactgagggtcaaactgcactaccagaagccagaggaatatgatcaactggtctggatggtggtcgggcaccaaggagaccactggaaggaagggcgtgtcttgctgcacaaatctctgaaactgtatcaggttatttttgaaggtgaaatcggaaaaggaaacctcggtgggattgctgtggatgatatcagtattaacaaccacattcctcaggaggactgtgcaaaaccaacagacctagataaaaagaacacagaaattaaaatagatgaaacagggagcaccccaggatatgaagaagggaaaggcgacaagaacatctccaggaagccaggcaatgtgcttaagaccctggaccccatcctgatcaccatcatagccatgagtgccctgggggtgctcctgggtgcagtctgtggagttgtgctgtactgtgcctgttggcacaatgggatgtcggaaaggaacctatctgccctggagaactataactttgaacttgtggatggtgtaaagttgaaaaaagataaactgaacccacagagtaattactcagaggcgtgaaggcacggagctggagggaacaagggaggagcgcggcaggagaacagtggaggcgcagggactctgttactctgctttcactgtaagctgggaagggcggggactctgttactccgctttcactgtaagctcggaagggcatccgcgatgccatgccaggcttttctcaggagcttcaatgagcatcacctacagacacaagcaggtgactgcggtaacaacaggaatcatgtacagcctgctttcttctcttggtttcgtttgggtaatcagaagccagttgagaccaagtgtgactgacttcatggttcatcctacttggccccctttttcctctctttctccttaccctgtggtggattcttctcggaaactgcaaaatccaagatgctggcactaggcgttgttcagtgggctctttcgatggacatgtgacctatagcccagtgcctagagcatattagcataaccacatttcaggggacaccaatgtccgcttttgcatcgctacgtgcagcgagcacaggaaaaaaaaaaaaaaaaaaaaaaaaaa

SEQ ID NO: 12 - protein, 922 aa, Rattus norvegicus
MERGLPLLCATLALALALAGAFRSDKCGGTIKIENPGYLTSPGYPHSYHPSEKCEWLIQAPEPYQRIMINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNSLECTYIIFAPKMSEIILEFESFDLEQDSNPPGGVFCRYDRLEIWDGFPEVGPHIGRYCGQKTPGRIRSSSGILSMVFYTDSAIAKEGFSANYSVLQSSISEDFKCMEALGMESGEIHSDQITASSQYGTNWSVERSRLNYPENGWTPGEDSYREWIQVDLGLLRFVTAVGTQGAISKETKKKYYVKTYRVDISSNGEDWITLKEGNKAIIFQGNTNPTDVVFGVFPKPLITRFVRIKPASWETGISMRFEVYGCKITDYPCSGMLGMVSGLISDSQITASNQGDRNWMPENIRLVTSRTGWALPPSPHPYINEWLQVDLGDEKIVRGVIIQGGKHRENKVFMRKFKIAYSNNGSDWKMIMDDSKRKAKSFEGNNNYDTPELRAFTPLSTRFIRIYPERATHSGLGLRMELLGCEVEVPTAGPTTPNGNPVDECDDDQANCHSGTGDDFQLTGGTTVLATEKPTIIDSTIQSEFPTYGFNCEFGWGSHKTFCHWEHDSHAQLRWRVLTSKTGPIQDHTGDGNFIYSQADENQKGKVARLVSPVVYSQSSAHCMTFWYHMSGSHVGTLRVKLHYQKPEEYDQLVWMVVGHQGDHWKEGRVLLHKSLKLYQVIFEGEIGKGNLGGIAVDDISINNHIPQEDCAKPTDLDKKNTEIKIDETGSTPGYEEGKGDKNISRKPGNVLKTLDPILITIIAMSALGVLLGAVCGVVLYCACWHNGMSERNLSALENYNFELVDGVKLKKDKLNPQSNYSEA

SEQ ID NO: 13 - DNA, 1739 bp, Rattus norvegicus
  CDS (100)..(1497)
  misc_feature (1719)..(1719): n is a, c, g, or t
ctatgcacattccttgtactcactgtgctgctgctgaggtgtgaggcaaactccgcttggggactgagctagtggagaaaggatcttaaaagcgacctgatgggttctgggaaagtcttccttttctctccttccctcctgtggagtcaaactagaggagtgaggttgatattcttgttactaaccctgcatctgggaaactgcattgataaagcagatgatgaagatgatgaagatttaactatgaacaaaacatgggtcttggcacctaaaattcatgaaggagatatcacacagattctcaactctttacttcaaggctatgataacaaacttcgtccagatataggcgtgagacccacagtaattgaaactgatgtttatgtaaacagcattggacctgttgatcccataaacatggagtacacgatagacatcatttttgcccagacttggttcgatagccgtttaaagttcaacagcaccatgaaagtccttatgcttaacagcaacatggtgggaaaaatttggatccctgacacgttcttcaggaactcaaggaaatctgatgcgcactggataacaacgcccaatcgcctgctgcggatatggagtgatggaagggttctctacactctgagattaacaattaatgcagaatgctaccttcaactccataactttcctatggatgaacattcctgtccactggaattttcaagctatggataccctaaaaatgaaattgagtacaagtggaaaaagccctcagtggaagtggctgatcctaagtactggagattgtatcagtttgcctttgtagggttaagaaattcaaccgaaatctctcacacaatctccggagattatattatcatgactatcttttttgacctgagcagacggatgggctatttcacaattcagacctacattccgtgcattctaacagttgttctttcctgggtgtctttttggatcaacaaagatgctgtacctgctagaacatccctgggtatcactacggttttgactatgacaaccctcagtacaatcgctagaaaatctttacctaaggtttcttacgtgacagcaatggatctctttgtctctgtgtgtttcatttttgtgtttgcagcactcatggagtatggaaccttgcattattttactagtaacaataaaggaaaaaccaccagagacaggaagctgaaaagcaaaacttcggtatccccaggtctccatgctggatctactctgattcccatgaacaacatttctatgcctcaaggggaagatgattatggttaccagtgcttggagggcaaagactgtgctacctttttctgttgctttgaagactgcagaactgggtcctggagagaagggcgaatacacatacgcattgccaaaatcgactcctactccaggatctttttcccaacagcttttgccttgttcaatctggtttactgggttggatatctatacttataagttctgccaatagataaaaaaattcataaaaatctggcttagtccaatataatcacttcgatttcataacaggaagttagaatttaaaatgaaagtctaccagttaaaatctgaatcatttggtcatatgattaaggctttcataaataaataagaaagaattattcaggttaatttacctaactaaacatgaaattcatgtccaatatagcacatttaaancagtaaacttaataattttt

SEQ ID NO: 14 - protein, 465 aa, Rattus norvegicus
MGSGKVFLFSPSLLWSQTRGVRLIFLLLTLHLGNCIDKADDEDDEDLTMNKTWVLAPKIHEGDITQILNSLLQGYDNKLRPDIGVRPTVIETDVYVNSIGPVDPINMEYTIDIIFAQTWFDSRLKFNSTMKVLMLNSNMVGKIWIPDTFFRNSRKSDAHWITTPNRLLRIWSDGRVLYTLRLTINAECYLQLHNFPMDEHSCPLEFSSYGYPKNEIEYKWKKPSVEVADPKYWRLYQFAFVGLRNSTEISHTISGDYIIMTIFFDLSRRMGYFTIQTYIPCILTVVLSWVSFWINKDAVPARTSLGITTVLTMTTLSTIARKSLPKVSYVTAMDLFVSVCFIFVFAALMEYGTLHYFTSNNKGKTTRDRKLKSKTSVSPGLHAGSTLIPMNNISMPQGEDDYGYQCLEGKDCATFFCCFEDCRTGSWREGRIHIRIAKIDSYSRIFFPTAFALFNLVYWVGYLYL

SEQ ID NO: 15 - DNA, 1946 bp, Rattus norvegicus
  CDS (131)..(1774)
aggatggaaatttccactctttaggaagcgtaaagaaaagaaggaagggtctctcactctcacccttgccgccacatccattggattccaacccgaagaaggtagaggcgggaaagggctgtgaggtcaaatggaccccatagaactgagaagtgtcaacatcgaaccatacgaggacagctgcagcgtggatagcattcaaagctgctacaccgggatggggaactccgagaagggagccatggacagtcaatttgctaatgaagatgccgaaagtcagaagttcctgacgaatggctttttggggaagaagacgctaaccgattacgcggatgagcaccaccctggaacgacctcttttggaatgtcctcctttaacctgagtaacgccatcatgggcagtggtatcttgggcttgtcctacgccatggccaacacggggatcgtcctttttgtaatcatgcttcttactgtggcaattctatcgctctactcggttcaccttttactgaagacagccaaggaaggagggtctttgatctatgaaaagttgggagagaaggcgtttggatggcctgggaaaattggagcctttatctctattacaatgcagaacattggagccatgtcaagctacctcttcatcattaagtacgagctgcctgaagtgatcagagtgttcatgggacttgaagaaaacactggggagtggtacctcaatggcaactacctcgtcttatttgtgtccgtggggatcatcctaccactctctctccttaaaaatttaggctaccttggctacaccagtggattttccctcacctgcatggtgtttttcgtcagtgtggtgatttacaaaaagttccaaataccctgccctctgcctgttctggatcacaataacgggaacctgacgttcaacaacacacttcccatgcacgtgatcatgctacccaataactcggagagcacgggcatgaacttcatggtggactacactcaccgagacccggaagggctggatgagaagccagccgcaggccctcttcacggcagtggtgtggagtacgaagcccacagtggggacaaatgtcaacccaagtactttgtattcaattcccgcacggcctacgcgattccaatcctggcgtttgctttcgtctgccaccctgaggtccttcccatctacagcgagcttaaggatcggtcccggagaaagatgcagacggtgtccaatatttccattaccgggatgctcgtcatgtacctgcttgctgcgctctttggctatctaagcttctacggggaagttgaggacgagttgctccatgcctacagtaaagtctacacatttgatacagctcttctcatggtacgcctggcagtgctggtggcggtgacactgactgtgcccatcgtcctgttccccatccgtacttcagtgatcacactgctgtttccaagaagacccttcagctgggtgaagcatttcgggatcgctgcaatcatcatcgctctcaacaacgtcctggtcatcctcgtgcctaccatcaaatacatctttggattcataggggcttcttctgccaccatgctgattttcattcttccggcagctttttatctcaagcttgtcaagaaagaaccccttcgatcaccccaaaagattggggctttggtcttcctcgtgactggaattatcttcatgatgggaagcatggcgctcattataatcgactggatttacaaccctccaaatcccgaccaccactaatccttgggggggatgctcttctattggaaacggccgacattatggtctgaaggacactttagttgtcttggttgggatgttagtctgaggaattaacgagattccgaagacattttctagctctatcatctgacgcatgtgaaaaaaaaaaaaaaaaaaaaaaaaaaaaaaa

SEQ ID NO: 16 - protein, 547 aa, Rattus norvegicus
MDPIELRSVNIEPYEDSCSVDSIQSCYTGMGNSEKGAMDSQFANEDAESQKFLTNGFLGKKTLTDYADEHHPGTTSFGMSSFNLSNAIMGSGILGLSYAMANTGIVLFVIMLLTVAILSLYSVHLLLKTAKEGGSLIYEKLGEKAFGWPGKIGAFISITMQNIGAMSSYLFIIKYELPEVIRVFMGLEENTGEWYLNGNYLVLFVSVGIILPLSLLKNLGYLGYTSGFSLTCMVFFVSVVIYKKFQIPCPLPVLDHNNGNLTFNNTLPMHVIMLPNNSESTGMNFMVDYTHRDPEGLDEKPAAGPLHGSGVEYEAHSGDKCQPKYFVFNSRTAYAIPILAFAFVCHPEVLPIYSELKDRSRRKMQTVSNISITGMLVMYLLAALFGYLSFYGEVEDELLHAYSKVYTFDTALLMVRLAVLVAVTLTVPIVLFPIRTSVITLLFPRRPFSWVKHFGIAAIIIALNNVLVILVPTIKYIFGFIGASSATMLIFILPAAFYLKLVKKEPLRSPQKIGALVFLVTGIIFMMGSMALIIIDWIYNPPNPDHH

SEQ ID NO: 17 - DNA, 1200 bp, Rattus norvegicus
  CDS (2)..(808)
catgagcgccgctcttttcagcctagacagcccagcacgcggcgcaccctggcccacagagcccgcggccttctacgagccaggcagggtgggcaagccaggacgagggccggagcctggggatctgggggagccgggctccacgacccctgccatgtatgacgacgagagcgccatcgacttcagcgcctacattgattccatggctgccgtgcccaccctagagttgtgccacgacgagatcttcgccgacctcttcaacagcaatcacaaagcggccggcgcgggcagcctggagctgctgcagggcggccctacgcgacccccgggtgtggggtcaatcgccaggggcccgctgaagcgcgaacccgactggggcgacggcgacgcgccgggctccctgctgccggcgcaagtggcagtgtgcgcgcagacagtggtgagcttggcggccgcggcacagcccacaccacccacttcgcccgagcctcctcgaggcagccctggaccgagccttgcgcctggccccgtccgagagaagggcgccggcaagaggggtccggaccggggcagccctgagtaccggcagcgacgcgagcgcaacaacatcgctgtgcgcaagagccgggacaaggccaagcgccgcaaccaggagatgcagcagaagctggtggagctgtcggccgagaacgagaagctgcatcagcgtgtggagcagctcacccgggacctggccagcctccggcagttcttcaaagagctgcccagcccgcctttcctgccgcccaccggcaccgactgccggtaacgcgcggtgtgggccttagagactccgaacgaccgatacctcagaccccgacggcggggagcagacgccgcccgaattgctacagtttcttgggcactggactgcgagagaagctatatgaatcccccttaaattatttttttataatggtagcgttttctacgtcttattaccattgcagctaaggtacattgtagaaaagacttttccgacagacttttgtagataagaggaagagactgcgcatgctttttatattcatttttacagtatttgtaagaataaagaagcatttaaattgcaaaaaaaaagaggcaccagctctgactggcctctttctaggctacggtgatcctgagcatcttttgttacctgctggtagaaatgatcct

SEQ ID NO: 18 - protein, 268 aa, Rattus norvegicus
MSAALFSLDSPARGAPWPTEPAAFYEPGRVGKPGRGPEPGDLGEPGSTTPAMYDDESAIDFSAYIDSMAAVPTLELCHDEIFADLFNSNHKAAGAGSLELLQGGPTRPPGVGSIARGPLKREPDWGDGDAPGSLLPAQVAVCAQTVVSLAAAAQPTPPTSPEPPRGSPGPSLAPGPVREKGAGKRGPDRGSPEYRQRRERNNIAVRKSRDKAKRRNQEMQQKLVELSAENEKLHQRVEQLTRDLASLRQFFKELPSPPFLPPTGTDCR

SEQ ID NO: 19 - DNA, 1237 bp, Rattus norvegicus
  CDS (151)..(648)
cgaggcagctgtgcagtgttccccagcgaggctaagcaagaagccggcaagagcagagacgcgagggtgggagccagcgcagagccggcgccgggcactgtgggggccaggagcagcccgcgcgccgcgtgagggactcgcacttgcaatatgactttggaggaattctcggccgcagagcagaagatcgaaaggatggacacggtgggcgatgccctggaggaagtgctcagcaaggctcggagtcagcgcaccataactgtcggcgtgtacgaggcagccaagctgctcaacgtagacccggacaacgtggtcctgtgcctgctggctgcggatgaagatgacgaccgggacgtggctctgcagatccatttcaccctcattcgtgctttctgttgcgagaacgacatcaacatcctgcgggtcagcaacccgggtcggctggcagagctgttgctactggagaacgacaagagccccgctgagagcgggggcgctgcgcagaccccggacttacactgtgtgctggtgacgaacccacattcatcacaatggaaggatcctgccttaagtcaacttatttgtttttgccgggaaagtcgctacatggatcagtgggtgccagtgattaatctccccgaacggtgattccccgaacggtgatggcatctgaatggaaataactgaaccaaattgcactgaagttttgaaatacctttgtagttactcaagcagtcactccccacgctgatgcaaggattacagaaactgatgtcaaggggctgagttcaactacaggagggctaggagatgactttgcagaaggagagagaggtgagactgaaggaggaagctgtgtggaaacagaaatccaagtcaaaagggacaaaaaaactacaaagaactgtgcaagaaagaaaactattaatttaggatggccgggttacagtaaagtaaaccaaatattgctttgttgaaactttaaatgtgtagcaatattttgggtattttttttggtcttcatgccctcaaataaaagaaaagtgaaaagggttaatcatattttcaagccacagtttaatgtattttgatgagatgttaaattctcagaagttttattataaatcgtactaagttattttatgatgtgaaaggttatttatgataaaacgtttttgaagcacattatctaaaataaactggtatggaataaaaaaaaaaaaaaaaaaaaaaaaaa

SEQ ID NO: 20 - protein, 165 aa, Rattus norvegicus
MTLEEFSAAEQKIERMDTVGDALEEVLSKARSQRTITVGVYEAAKLLNVDPDNVVLCLLAADEDDDRDVALQIHFTLIRAFCCENDINILRVSNPGRLAELLLLENDKSPAESGGAAQTPDLHCVLVTNPHSSQWKDPALSQLICFCRESRYMDQWVPVINLPER

SEQ ID NO: 21 - DNA, 21 bp, Artificial Sequence
  Primer
ccctcagagtctgctggatca

SEQ ID NO: 22 - DNA, 21 bp, Artificial Sequence
  Primer
aggctacaagtggctgcttca

SEQ ID NO: 23 - DNA, 22 bp, Artificial Sequence
  Primer
ccttgcgaggagtaaagcatgt

SEQ ID NO: 24 - DNA, 20 bp, Artificial Sequence
  Primer
catcgagcacaatgggacaa

SEQ ID NO: 25 - DNA, 29 bp, Artificial Sequence
  Primer
gtagaagttataataggagacaaccacat

SEQ ID NO: 26 - DNA, 26 bp, Artificial Sequence
  Primer
aaacgaggataagagttgttctccat

SEQ ID NO: 27 - DNA, 20 bp, Artificial Sequence
  Primer
ctccactggccaatgtcaaa

SEQ ID NO: 28 - DNA, 21 bp, Artificial Sequence
  Primer
ggagaacctcaggagcgagat

SEQ ID NO: 29 - DNA, 31 bp, Artificial Sequence
  Primer
ntttcgggcaatgtagatcaccttgaccttn

SEQ ID NO: 30 - DNA, 29 bp, Artificial Sequence
  Primer
naggctgtacaccctcttctgcgctgtgn

SEQ ID NO: 31 - DNA, 24 bp, Artificial Sequence
  Primer
ntcctccttccctcggacctgcan

SEQ ID NO: 32 - DNA, 24 bp, Artificial Sequence
  Primer
nccaccttctgtggcacgcctgan

(19) United States
(12) Patent Application Publication
(10) Pub. No.: US 2010/0040552 A1
(43) Pub. Date: Feb. 18, 2010

(54) METHODS TO EVALUATE GLUCOCORTICOID RECEPTOR AGONISTS AND ANTAGONISTS FOR EFFECTS ON NEURON-LIKE CELLS

(75) Inventors: David J. Stone, Wyncote, PA (US); Janet E. Clark, Matawan, NJ (US); Edward C. Hayes, III, Brick, NJ (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: ROSETTA INPHARMATICS LLC, Seattle, WA (US); MERCK & CO., INC., Rahway, NJ (US)

(21) Appl. No.: 12/579,230

(22) Filed: Oct. 14, 2009

Related U.S. Application Data
(63) Continuation of application No. 11/540,482, filed on Sep. 27, 2006.
(60) Provisional application No. 60/721,051, filed on Sep. 27, 2005.

(51) Int. Cl. A61K 49/00; G01N 33/53; A61P 29/00

(52) U.S. Cl. 424/9.2; 435/7.21

(57) ABSTRACT

The present invention provides methods for identifying a modulator of glucocorticoid receptor activity. In one embodiment, the methods include the steps of (a) contacting neuron-like cells, in vitro, with a chemical agent; (b) measuring the expression of a member, or group of members, of a group of genes (as defined herein) in the neuron-like cells contacted with the chemical agent; and (c) determining whether the chemical agent significantly alters the expression of the member, or group of members, of the group of genes, thereby determining whether the chemical agent is likely to be a modulator of glucocorticoid receptor activity. In another embodiment, an in vivo method for identifying a modulator of glucocorticoid receptor activity is provided.

### CROSS-REFERENCES TO RELATED APPLICATIONS

This application is a continuation of U.S. application Ser. No. 11/540,482, filed Sep. 27, 2006, which claims the benefit of U.S. Provisional Application No. 60/721,051, filed Sep. 27, 2005, the disclosures of which are hereby expressly incorporated herein in their entirety.

### FIELD OF THE INVENTION

The present invention relates to methods for identifying drugs that are useful for treating psychiatric disorders and/or inflammation.

### BACKGROUND

The regulation of stress hormones by the hypothalamic-pituitary-adrenal axis (HPA) has been implicated in the pathophysiology of several psychiatric disorders and there is clinical evidence that glucocorticoid receptor (GR) antagonists may ameliorate many of the symptoms of these conditions. Studies on the use of mifepristone (a GR modulator identified as RU-486) to treat psychotic major depression have shown encouraging results (Belanoff et al., “Rapid Reversal of Psychotic Depression Using Mifepristone,” J. Clin. Psychopharmacol. 21(5):516-521, October 2001; Belanoff et al., “An Open Label Trial of C-1073 (Mifepristone) for Psychotic Major Depression,” Biol. Psychiatry 52(5):386-392, September 2002). Mood showed significant improvements, especially at higher doses. Similarly, mifepristone also decreased scores on both the overall Brief Psychiatric Rating Scale (BPRS) and on the BPRS positive symptom subscale, suggesting an overall lessening of psychotic symptoms (Belanoff et al., 2002, supra). In a double blind crossover study in bipolar disorder, mifepristone increased spatial working memory and had positive effects on verbal memory and mood (Young et al., “Improvements in Neurocognitive Function and Mood Following Adjunctive Treatment With Mifepristone (RU-486) in Bipolar Disorder,” Neuropsychopharmacology 29(8):1538-1545, August 2004). Glucocorticoid receptor modulators may, therefore, have cognitive enhancing, antidepressant, and antipsychotic properties. Consequently, glucocorticoid receptor modulators are a potential class of drugs for the treatment of psychiatric disorders.

### SUMMARY

In one aspect, the present invention provides in vitro methods to identify modulators of glucocorticoid receptor activity in neuron-like cells that have phenotypic characteristics of serotonergic neurons (i.e., neurons that synthesize serotonin) that are known to play a role in some psychiatric diseases. In the practice of one embodiment of the invention, the levels and/or pattern of expression of one or more members of a group of ten genes, as described more fully herein, is/are measured in the neuron-like cells in response to a chemical agent. Chemical agents that are modulators of the glucocorticoid receptor, which is implicated in various psychiatric diseases, cause a change in the expression of one or more of these genes. These genes have been associated in the scientific literature with schizophrenia, bipolar disorder, anxiety, and/or hippocampal spine density. Three of these genes (RGS2, Sult1a1, and RGS4) are regulated, in vivo, in the rat caudate and/or raphe nuclei after administration of the glucocorticoid receptor agonists prednisolone and dexamethasone, as further described in Example 1. Moreover, Sult1a1 gene expression is upregulated in vivo in the dorsal hippocampus in response to dexamethasone, and reversal of this effect occurs when RU-486 is co-administered with dexamethasone, as further described in Example 2.

In one embodiment, the present invention provides methods for identifying potential modulators of glucocorticoid receptor activity. The methods of this aspect of the invention each includes the steps of (a) contacting neuron-like cells, in vitro, with a chemical agent; (b) measuring the expression of a member, or group of members, of the group of genes consisting of Sult1a1, RGS2, RGS4, IL6R, SGK, neuropilin, Gabrg1, Ata3, Cebpd, and GADD45a in the neuron-like cells contacted with the chemical agent; and (c) determining whether the chemical agent significantly alters the expression of the member, or group of members, of the group of genes consisting of Sult1a1, RGS2, RGS4, IL6R, SGK, neuropilin, Gabrg1, Ata3, Cebpd, and GADD45a, wherein a significant alteration in expression relative to neuron-like cells not contacted with the chemical agent is indicative of the chemical agent possessing the ability to modulate glucocortocoid receptor activity.

Thus, if the chemical agent significantly alters the expression of one, or several (e.g., 2, 3, 4, 5, 6, 7, 8, 9, or 10), member(s) of the group of genes consisting of Sult1a1, RGS2, RGS4, IL6R, SGK, neuropilin, Gabrg1, Ata3, Cebpd, and GADD45a, then the chemical agent is likely to be a modulator of glucocorticoid receptor activity. Conversely, if the chemical agent does not significantly alter the expression of one, or several (e.g., 2, 3, 4, 5, 6, 7, 8, 9, or 10), member(s) of the group of genes consisting of Sult1a1, RGS2, RGS4, IL6R, SGK, neuropilin, Gabrg1, Ata3, Cebpd, and GADD45a, then the chemical agent is not likely to be a modulator of glucocorticoid receptor activity. If the chemical agent significantly alters the expression of one, or several, of the aforementioned genes and is therefore likely to be a modulator of glucocorticoid receptor activity, then the ability of the chemical agent to modulate glucocorticoid receptor activity can be confirmed by using an in vivo assay that measures the effect of the chemical agent on a biological response that is mediated by a glucocorticoid receptor. Examples of biological responses that are mediated by a glucocorticoid receptor are described herein (e.g., changes in the expression of mRNAs involved in GABA production).

In a further aspect, the present invention provides methods for identifying potential antagonists of glucocorticoid receptor activity, the method comprising the steps of (a) comparing the expression of a member, or group of members, of a group of genes in neuron-like cells contacted with a chemical agent and a glucocorticoid receptor agonist, with the expression of the member, or group of members, of the group of genes in neuron-like cells contacted with the glucocorticoid receptor agonist but not with the chemical agent, wherein the group of genes consists of Sult1a1, RGS2, RGS4, IL6R, SGK, neuropilin, Gabrg1, Ata3, Cebpd, and GADD45a; and (b) determining whether the chemical agent prevents or reverses a change in gene expression, caused by the glucocorticoid receptor agonist, of the member, or the group of members, of the group of genes, thereby determining whether the chemical agent is likely to be an antagonist of glucocorticoid receptor activity. Thus, if the chemical agent prevents, or reverses, a change in gene expression, caused by the glucocorticoid receptor agonist, of one, or more, member(s) of the group of genes, then the chemical agent is likely to be an antagonist of glucocorticoid receptor activity. Examples of useful glucocorticoid receptor agonists include dexamethasone, prednisolone and corticosterone.

If the chemical agent significantly alters the expression of one or several of the aforementioned genes and is therefore likely to be an antagonist of glucocorticoid receptor activity, then the ability of the chemical agent to antagonize glucocorticoid receptor activity can be confirmed by using an in vivo assay that measures the effect of the chemical agent on a biological response that is mediated by a glucocorticoid receptor. The methods of the present invention can be used to identify modulators of glucocorticoid receptor activity.

In another aspect, the present invention provides in vivo methods for identifying potential modulators of glucocorticoid receptor activity. The methods of this aspect of the invention include the steps of (a) administering a chemical agent to a test animal; and (b) measuring the expression of a member, or group of members, of the group of genes consisting of Sult1a1, RGS2, RGS4, IL6R, SGK, neuropilin, Gabrg1, Ata3, Cebpd, and GADD45a in tissue obtained from the test animal administered with the chemical agent; wherein a change in expression of a member, or group of members of the group consisting of Sult1a1, RGS2, RGS4, IL6R, SGK, neuropilin, Gabrg1, Ata3, Cebpd, and GADD45a relative to an animal not administered the chemical agent is indicative of the chemical agent possessing the ability to modulate glucocortocoid receptor activity in vivo. The methods of this aspect of the invention can be used to identify modulators of glucocorticoid receptor activity, and may also be used to confirm results obtained from an in vitro screening method as described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the effect of 6α-methylprednisolone on the expression of genes RGS2 and RGS4 in the rat caudate nucleus, as described in Example 1 herein. The following abbreviations are used in FIG. 1: 6α-MP, 6α-methylprednisolone; V, vehicle (the solution used to dissolve 6α-methylprednisolone—the vehicle treatment is the control treatment); mpk, milligrams per kilogram body weight (used to express dosage of 6α-MP administered to rats). Cross-hatched bars represent RGS2 gene expression. Unhatched bars represent RGS4 gene expression. ** p=0.003. * p=0.001.

FIG. 2 shows the effect of dexamethasone on the expression of gene Sult1a1 in the rat caudate nucleus and raphe nucleus, as described in Example 1 herein. The following abbreviations are used in FIG. 2: Dex, dexamethasone; V, vehicle (the solution used to dissolve dexamethasone—the vehicle treatment is the control treatment); mpk, milligrams per kilogram body weight (used to express dosage of dexamethasone administered to rats). Cross-hatched bars represent Sult1a1 gene expression in the caudate nucleus. Unhatched bars represent Sult1a1 gene expression in the raphe nucleus. *p<0.001.

### DETAILED DESCRIPTION

In one aspect, the present invention provides methods for identifying potential modulators of glucocorticoid receptor activity. The methods of this aspect of the invention each include the steps of (a) contacting neuron-like cells, in vitro, with a chemical agent; (b) measuring the expression of a member, or group of members, of the group of genes consisting of Sult1a1, RGS2, RGS4, IL6R, SGK, neuropilin, Gabrg1, Ata3, Cebpd, and GADD45a in the neuron-like cells contacted with the chemical agent; and (c) determining whether the chemical agent significantly alters the expression of the member, or group of members, of the group of genes consisting of Sult1a1, RGS2, RGS4, IL6R, SGK, neuropilin, Gabrg1, Ata3, Cebpd, and GADD45a, wherein a significant alteration in expression relative to neuron-like cells not contacted with the chemical agent is indicative of the chemical agent possessing the ability to modulate glucocortocoid receptor activity.

The glucocorticoid receptor is a steroid hormone receptor that binds cortisol. The complex of glucocorticoid receptor and bound cortisol binds to the promoter element of a target gene. The glucocorticoid receptor, like all steroid receptors, is a zinc-finger transcription factor. Glucocorticoid receptors are described, for example, in Endocrinology: Basic and Clinical Principles, Chapter 4, P. M. Conn and S. Melmed, Eds., Humana Press Inc., Totowa, N.J. 1997.

The methods of the present invention can be used to determine whether any type of chemical agent is a potential modulator of glucocorticoid receptor activity. For example, the methods of the present invention can be used to determine whether a chemical element, a chemical compound (e.g., a steroid), or a combination of chemical compounds and/or elements is a potential modulator of glucocorticoid receptor activity. The term “chemical agent” includes candidate therapeutic molecules that may be useful for treating one or more diseases in a living organism.

A modulator of glucocorticoid receptor activity is a chemical agent (or combination of agents) that changes the activity of a glucocorticoid receptor. For example, a modulator of glucocorticoid receptor activity may increase or decrease the activity of a glucocorticoid receptor. Glucocorticoid receptor molecules are components of signal transduction pathways that mediate numerous biological responses. Thus, the activity of a glucocorticoid receptor can be measured, for example, by measuring one or more biological responses that is/are controlled by a signal transduction pathway that includes a glucocorticoid receptor.

For example, administration of a glucocorticoid receptor agonist drastically alters the hippocampal GABA system, changing the expression of mRNAs involved in GABA production (e.g., GAD65 and GAD67), and GABA binding (GABA_{A} receptor subunits), and the binding of benzodiazepine to the GABA_{A} receptor (see, Stone, D. J., et al., “Effects of Pre- and Post-Natal Corticosterone Exposure on the Rat Hippocampal GABA System,” Hippocampus. 11(5):492-507, 2001). This effect is of interest in psychiatric diseases because the hippocampal GABA system is altered in the brains of schizophrenic and bipolar disorder patients (Heckers, S., et al., “Differential Hippocampal Expression of Glutamic Acid Decarboxylase 65 and 67 Messenger RNA in Bipolar Disorder and Schizophrenia,” Arch. Gen. Psychiatry. 59(6):521-529, June 2002).

Glucocorticoid receptors are also involved in the regulation of at least some inflammatory responses. While not wishing to be bound by theory, the effect of glucocorticoid receptors on inflammation may be due to transrepression of proinflammatory factors, whereas transactivation of inappropriate genes involved in metabolic function is thought to lead to glucocorticoid-induced diabetes and osteoporosis. Genes known to be transactivated by glucocorticoid receptors include genes encoding glucose-6-phosphatase, phosphoenolpyruvatecarboxykinase, tyrosine aminotransferase, secretory leukoprotease inhibitor and the β2-adrenoreceptor. Genes known to be transrepressed encode propiomelanocortin (POMC), corticotrophin releasing hormone (CRH), prolactin, osteocalcin and inflammatory cytokines. (see, e.g., “The Pursuit of Differentiated Ligands for the Glucocorticoid Receptor,” Coghlan, M. J., et al., Curr. Topics Med. Chem. 3:1617-1635, 2003).

The term “neuron-like cells” refers to cells that express neuron-specific enolase and neurofilaments that have neurites of at least 100 μm in length, and that express neural markers such as synaptophysin or neural-type ion channels. An example of neuron-like cells are CA77 cells described by Muszynski et al. (“Glucocorticoids Stimulate the Production of Preprocalcitonin-Derived Secretory Peptides by a Rat Medullary Thyroid Carcinoma Cell Line,” J. Biol. Chem. 258(19):11678-11683, October 1983), which publication is incorporated herein by reference.

An example of a method for creating a cultured population of neuron-like cells, in vitro, is disclosed by Muszynski et al., supra. Muszynski et al. discloses methods for selecting and culturing a medullary thyroid carcinoma cell line (which is a type of neuron-like cell line). In brief, a medullary thyroid carcinoma is selected wherein the cancerous cells express neurofilaments and neuron-specific enolase. These cells are serially passaged and cells are selected to establish a cell line that has some, preferably all, of the following properties: The presence of neurites of at least 100 μm in length; the presence of neurofilaments; expression of neuron-like markers, such as synaptophysin, and neural-type ion channels. The selected cells also preferably exhibit a serotinergic phenotype (e.g., serotonin immunostaining, storage and release, and the expression of serotonin autoreceptors and transporters). The selected cell line must express a glucocorticoid receptor and be responsive to glucocorticoid agonists such as dexamethasone and corticosterone.

Neuron-like cells are typically cultured in a liquid medium and can be contacted with a chemical agent, for example, by adding the chemical agent to the liquid culture medium. Representative culture conditions for neuron-like cells are described, for example, in Muszynski, M., et al., supra, and in L. A. Tverberg and A. F. Russo, “Cell-Specific Glucocorticoid Repression of Calcitonin/Calcitonin Gene-Related Peptide Transcription Localization to an 18-Base Pair Basal Enhancer Element,” J. Biol. Chem. 267:17567-17573, 1992, which publications are incorporated herein by reference.

In the practice of the present invention, the expression of at least one member of the group of genes consisting of Sult1a1, RGS2, RGS4, IL6R, SGK, neuropilin, Gabrg1, Ata3, Cebpd, and GADD45a is measured. Typically the expression of several, or all, members of the group of ten genes consisting of Sult1a1, RGS2, RGS4, IL6R, SGK, neuropilin, Gabrg1, Ata3, Cebpd, and GADD45a is measured. The expression of a member, or members, of the foregoing group of ten genes is measured by measuring the absolute or relative amounts (and/or changes in the absolute or relative amounts) of mRNA molecules (or nucleic acid molecules derived therefrom) transcribed from a member, or members, of the foregoing group of ten genes. Thus, when used with reference to a member of the group of genes consisting of Sult1a1, RGS2, RGS4, IL6R, SGK, neuropilin, Gabrg1, Ata3, Cebpd, and GADD45a, the term “transcript” or “gene transcript” includes mRNA transcribed from one or more of the foregoing ten genes, and also includes nucleic acid molecules (e.g., cDNA or cRNA) derived from mRNA transcribed from one or more of the foregoing 10 genes. Thus, by way of specific example, the term “transcript” or “gene transcript” encompasses cDNA that is complementary to mRNA transcribed from a member of the group of genes consisting of Sult1a1, RGS2, RGS4, IL6R, SGK, neuropilin, Gabrg1, Ata3, Cebpd, and GADD45a.

Sult1a1 is the abbreviation for Sulfotransferase lal. Sult1a1 inactivates dopamine by sulfation and the Sult1a1 gene is located at 16p12.1-p11.2, near a locus linked to bipolar disorder (Ewald, H. et al., “A Possible Locus for Manic Depressive Illness on Chromosome 16p13,” Psychiatr. Genet. 5(2):71-81, 1995; McInnis, M. G., et al., “Genome-Wide Scan and Conditional Analysis in Bipolar Disorder: Evidence for Genomic Interaction in the National Institute of Mental Health Genetics Initiative Bipolar Pedigrees,” Biol. Psychiatry 54(11):1265-1273, December 2003). An exemplary Sult1a1 cDNA sequence is set forth in GenBank accession number NM_{—}031834 (SEQ ID NO:1). Sult1a1 genes useful in the practice of the present invention encode a Sult1a1 protein (e.g., the Sult1a1 protein having the amino acid sequence set forth in SEQ ID NO:2, which is encoded by the cDNA having the nucleic acid sequence set forth in SEQ ID NO:1). In some embodiments, the Sult1a1 protein is at least 95% identical (e.g., at least 96% identical, or at least 97% identical, or at least 98% identical, or at least 99% identical) to the Sult1a1 protein having the amino acid sequence set forth in SEQ ID NO:2. In some embodiments, Sult1a1 gene transcripts that are measured in the practice of the present invention are at least 95% identical (e.g., at least 96% identical, or at least 97% identical, or at least 98% identical, or at least 99% identical) to the Sult1a1 cDNA sequence set forth in SEQ ID NO:1.

RGS2 is the abbreviation for Regulator of G-protein signaling 2. RGS2 decreases Gα_{q} signaling by the 5-HT2A receptor and its mRNA is up-regulated by haloperidol (Taymans et al., “Dopamine Receptor-Mediated Regulation of RGS2 and RGS4 mRNA Differentially Depends on Ascending Dopamine Projections and Time,” Eur. J. Neurosci. 19(8):2249-2260, April 2004). An exemplary RGS2 cDNA sequence is set forth in GenBank accession number NM_{—}053453 (SEQ ID NO:3). RGS2 genes useful in the practice of the present invention encode an RGS2 protein (e.g., the RGS2 protein having the amino acid sequence set forth in SEQ ID NO:4, which is encoded by the cDNA having the nucleic acid sequence set forth in SEQ ID NO:3). In some embodiments, the RGS2 protein is at least 95% identical (e.g., at least 96% identical, or at least 97% identical, or at least 98% identical, or at least 99% identical) to the RGS2 protein having the amino acid sequence set forth in SEQ ID NO:4. In some embodiments, RGS2 gene transcripts that are measured in the practice of the present invention are at least 95% identical (e.g., at least 96% identical, or at least 97% identical, or at least 98% identical, or at least 99% identical) to the RGS2 cDNA sequence set forth in SEQ ID NO:3.

RGS4 is the abbreviation for Regulator of G-protein signaling 4. RGS4 decreases Gα_{q} signaling by the 5-HT1A and 5-HT2A receptors and shows down-regulation of mRNA levels in response to haloperidol (Taymans et al., “Dopamine Receptor-Mediated Regulation of RGS2 and RGS4 mRNA Differentially Depends on Ascending Dopamine Projections and Time,” Eur. J. Neurosci. 19(8):2249-2260, April 2004). An exemplary RGS4 cDNA sequence is set forth in GenBank accession number NM_{—}017214 (SEQ ID NO:5). RGS4 genes useful in the practice of the present invention encode an RGS4 protein (e.g., the RGS4 protein having the amino acid sequence set forth in SEQ ID NO:6, which is encoded by the cDNA having the nucleic acid sequence set forth in SEQ ID NO:5). In some embodiments, the RGS4 protein is at least 95% identical (e.g., at least 96% identical, or at least 97% identical, or at least 98% identical, or at least 99% identical) to the RGS4 protein having the amino acid sequence set forth in SEQ ID NO:6. In some embodiments, RGS4 gene transcripts that are measured in the practice of the present invention are at least 95% identical (e.g., at least 96% identical, or at least 97% identical, or at least 98% identical, or at least 99% identical) to the RGS4 cDNA sequence set forth in SEQ ID NO:5.

IL6R is the abbreviation for Interleukin 6 Receptor. IL6 is a neuromodulator that may have neurotrophic effects; and expression is induced in neurons by excitatory amino acids. (Juttler E., V. Tarabin, and M. Schwaninger, “Interleukin-6 (IL-6): A Possible Neuromodulator Induced by Neuron-Like Activity,” Neuroscientist. 8(3):268-275, June 2002). An exemplary IL6R cDNA sequence is set forth in GenBank accession number NM_{—}017020 (SEQ ID NO:7). IL6R genes useful in the practice of the present invention encode an IL6R protein (e.g., the IL6R protein having the amino acid sequence set forth in SEQ ID NO:8, which is encoded by the cDNA having the nucleic acid sequence set forth in SEQ ID NO:7). In some embodiments, the IL6R protein is at least 95% identical (e.g., at least 96% identical, or at least 97% identical, or at least 98% identical, or at least 99% identical) to the IL6R protein having the amino acid sequence set forth in SEQ ID NO:8. In some embodiments, IL6R gene transcripts that are measured in the practice of the present invention are at least 95% identical (e.g., at least 96% identical, or at least 97% identical, or at least 98% identical, or at least 99% identical) to the IL6R cDNA sequence set forth in SEQ ID NO:7.

SGK is the abbreviation for Serum/Glucocorticoid regulated kinase. SGK is expressed in the brain and is believed to be involved in memory formation (Lee, E. H., et al., “Enrichment Enhances the Expression of SGK, a Glucocorticoid-Induced Gene, and Facilitates Spatial Learning Through Glutamate AMPA Receptor Mediation,” Eur. J. Neurosci. 18(10):2842-2852 (November, 2003)). An exemplary SGK cDNA sequence is set forth in GenBank accession number NM_{—}019232 (SEQ ID NO:9). SGK genes useful in the practice of the present invention encode an SGK protein (e.g., the SGK protein having the amino acid sequence set forth in SEQ ID NO:10, which is encoded by the cDNA having the nucleic acid sequence set forth in SEQ ID NO:9). In some embodiments, the SGK protein is at least 95% identical (e.g., at least 96% identical, or at least 97% identical, or at least 98% identical, or at least 99% identical) to the SGK protein having the amino acid sequence set forth in SEQ ID NO:10. In some embodiments, SGK gene transcripts that are measured in the practice of the present invention are at least 95% identical (e.g., at least 96% identical, or at least 97% identical, or at least 98% identical, or at least 99% identical) to the SGK cDNA sequence set forth in SEQ ID NO:9.

Neuropilin is a VEGF receptor that is involved in axon guidance and neurogenesis. An exemplary neuropilin cDNA sequence is set forth in GenBank accession number NM_{—}145098 (SEQ ID NO:11). Neuropilin genes useful in the practice of the present invention encode a neuropilin protein (e.g., the neuropilin protein having the amino acid sequence set forth in SEQ ID NO:12, which is encoded by the cDNA having the nucleic acid sequence set forth in SEQ ID NO:11). In some embodiments, the neuropilin protein is at least 95% identical (e.g., at least 96% identical, or at least 97% identical, or at least 98% identical, or at least 99% identical) to the neuropilin protein having the amino acid sequence set forth in SEQ ID NO:12. In some embodiments, neuropilin gene transcripts that are measured in the practice of the present invention are at least 95% identical (e.g., at least 96% identical, or at least 97% identical, or at least 98% identical, or at least 99% identical) to the neuropilin cDNA sequence set forth in SEQ ID NO:11.

Gabrg1 is the abbreviation for gamma-aminobutyric acid (GABA) A receptor, gamma 1. Gabrg1 is a subunit of the GABAA receptor, an ion channel receptor for GABA, the major inhibitory neurotransmitter in the brain. An exemplary Gabrg1 cDNA sequence is set forth in GenBank accession number NM_{—}080586 (SEQ ID NO:13). Gabrg1 genes useful in the practice of the present invention encode a Gabrg1 protein (e.g., the gabrg1 protein having the amino acid sequence set forth in SEQ ID NO:14, which is encoded by the cDNA having the nucleic acid sequence set forth in SEQ ID NO:13). In some embodiments, the Gabrg1 protein is at least 95% identical (e.g., at least 96% identical, or at least 97% identical, or at least 98% identical, or at least 99% identical) to the Gabrg1 protein having the amino acid sequence set forth in SEQ ID NO:14. In some embodiments, Gabrg1 gene transcripts that are measured in the practice of the present invention are at least 95% identical (e.g., at least 96% identical, or at least 97% identical, or at least 98% identical, or at least 99% identical) to the Gabrg1 cDNA sequence set forth in SEQ ID NO:13.

Slc38a4 (also known as Ata3) is the abbreviation for solute carrier 38a4 (or amino acid transport system A3). Sc138a3/Ata3 is believed to be a transporter for alpha-(methylamino) isobutyric acid). An exemplary Ata3 cDNA sequence is set forth in GenBank accession number NM_{—}130748 (SEQ ID NO:15). Ata3 genes useful in the practice of the present invention encode an Ata3 protein (e.g., the Ata3 protein having the amino acid sequence set forth in SEQ ID NO:16, which is encoded by the cDNA having the nucleic acid sequence set forth in SEQ ID NO:15). In some embodiments, the S1c38a4 protein is at least 95% identical (e.g., at least 96% identical, or at least 97% identical, or at least 98% identical, or at least 99% identical) to the Slc38a4 protein having the amino acid sequence set forth in SEQ ID NO:16. In some embodiments, Slc38a4 gene transcripts that are measured in the practice of the present invention are at least 95% identical (e.g., at least 96% identical, or at least 97% identical, or at least 98% identical, or at least 99% identical) to the Slc38a4 cDNA sequence set forth in SEQ ID NO:15.

Cebpd is the abbreviation for CCAAT/enhancer binding protein (C/EBP), delta. Cebpd is an enhancer of C/EBP-mediated RNA transcription. An exemplary Cebpd cDNA sequence is set forth in GenBank accession number NM_{—}013154 (SEQ ID NO:17). Cebpd genes useful in the practice of the present invention encode a Cebpd protein (e.g., the Cebpd protein having the amino acid sequence set forth in SEQ ID NO:18, which is encoded by the cDNA having the nucleic acid sequence set forth in SEQ ID NO:17). In some embodiments, the Cebpd protein is at least 95% identical (e.g., at least 96% identical, or at least 97% identical, or at least 98% identical, or at least 99% identical) to the Cebpd protein having the amino acid sequence set forth in SEQ ID NO:18. In some embodiments, Cebpd gene transcripts that are measured in the practice of the present invention are at least 95% identical (e.g., at least 96% identical, or at least 97% identical, or at least 98% identical, or at least 99% identical) to the Cebpd cDNA sequence set forth in SEQ ID NO:17.

GADD45a is the abbreviation for growth arrest and DNA-damage-inducible 45 alpha. GADD45a mediates a delay in G2 to M cell cycle progression and also may induce DNA repair. An exemplary GADD45a cDNA sequence is set forth in GenBank accession number NM_{—}024127 (SEQ ID NO:19). GADD45a genes useful in the practice of the present invention encode a GADD45a protein (e.g., the GADD45a protein having the amino acid sequence set forth in SEQ ID NO:20, which is encoded by the cDNA having the nucleic acid sequence set forth in SEQ ID NO:19). In some embodiments, the GADD45a protein is at least 95% identical (e.g., at least 96% identical, or at least 97% identical, or at least 98% identical, or at least 99% identical) to the GADD45a protein having the amino acid sequence set forth in SEQ ID NO:20. In some embodiments, GADD45a gene transcripts that are measured in the practice of the present invention are at least 95% identical (e.g., at least 96% identical, or at least 97% identical, or at least 98% identical, or at least 99% identical) to the GADD45a cDNA sequence set forth in SEQ ID NO:19.

Sequence identity (typically expressed as percent identity) in the context of two nucleic acid sequences, or two amino acid sequences, refers to the number of nucleic acid residues, or amino acid residues, in the two sequences that are the same when the two sequences are aligned for maximum correspondence over a specified comparison window. Sequence identity values provided herein refer to the value obtained using GAP (e.g., GCG programs (Accelrys, Inc., San Diego, Calif.) version 10) using the default parameters. GAP uses the algorithm of Needleman & Wunsch, J. Mol. Biol. 48:443-453, 1970, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases.

An equivalent method to GAP may be used. The term “equivalent method” refers to any sequence comparison method, such as a sequence comparison program that, for any two sequences in question, generates an alignment having identical nucleic acid residue matches or identical amino acid residue matches, and an identical percent sequence identity when compared to the corresponding alignment generated by GAP.

The absolute or relative amounts of expression of one or more genes, or the pattern of expression of multiple genes, can be measured by any method for measuring gene expression. For example, reverse transcription followed by PCR (referred to as RT-PCR) can be used to measure gene expression. RT-PCR involves the PCR amplification of a reverse transcription product, and can be used, for example, to amplify very small amounts of any kind of RNA (e.g., mRNA, rRNA, tRNA). RT-PCR is described, for example, in Chapters 6 and 8 of The Polymerase Chain Reaction, Mullis, K. B., et al., Eds., Birkhauser, 1994, the cited chapters of which publication are incorporated herein by reference.

Again by way of example, ArrayPlate™ kits (sold by High Throughput Genomics, Inc., 6296 E. Grant Road, Tucson, Ariz. 85712) can be used to measure gene expression. In brief, the ArrayPlate™ mRNA assay combines a nuclease protection assay with array detection. Cells in microplate wells are subjected to a nuclease protection assay. Cells are lysed in the presence of probes that bind targeted mRNA species. Upon addition of S1 nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another ArrayPlate™ called a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells. The ArrayPlate™ technology is described in Martel, R. R., et al., Assay and Drug Development Technologies 1(1):61-71, 2002, which publication is incorporated herein by reference.

By way of further example, DNA microarrays can be used to measure gene expression. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, BioEssays 18:427, 1996). Exemplary microarrays and methods for their manufacture and use are set forth in T. R. Hughes et al., Nature Biotechnology 19:342-347, April 2001, which publication is incorporated herein by reference.

Art-recognized statistical techniques can be used to compare the levels of expression of individual genes, or groups of genes, to identify genes which exhibit statistically significantly different expression levels in neuron-like cells that have been contacted with a chemical agent compared to control neuron-like cells that are not contacted with the chemical agent. Thus, for example, a t-test can be used to determine whether the mean value of repeated measurements of the level of expression of a particular gene is significantly different in neuron-like cells that have been contacted with a chemical agent compared to control neuron-like cells that are not contacted with the chemical agent. Similarly, Analysis of Variance (ANOVA) can be used to compare the mean values of two or more populations of genes (e.g., two or more populations of genes in neuron-like cells that have been contacted with a chemical agent, compared to control neuron-like cells that are not contacted with the chemical agent) to determine whether the means are significantly different.

The following publications describe examples of art-recognized statistical techniques that can be used to compare the levels of expression of individual genes in neuron-like cells that have been contacted with a chemical agent compared to control neuron-like cells that are not contacted with the chemical agent, to identify genes that exhibit significantly different expression levels in response to a chemical agent: Nature Genetics 32(Suppl.):461-552, December 2002; Bioinformatics 18(4):546-554, April 2002; Dudoit et al., Technical Report 578, University of California at Berkeley; Tusher et al., Proc. Nat'l. Acad. Sci. USA 98(9):5116-5121, April 2001; and Kerr, et al., J. Comput. Biol. 7:819-837.

In a further aspect, the present invention provides methods for identifying potential antagonists of glucocorticoid receptor activity. The methods of this aspect of the invention each include the steps of (a) comparing the expression of a member, or group of members, of a group of genes in neuron-like cells contacted with a chemical agent and a glucocorticoid receptor agonist, with the expression of the member, or group of members, of the group of genes in neuron-like cells contacted with the glucocorticoid receptor agonist but not with the chemical agent, wherein the group of genes consists of Sult1a1, RGS2, RGS4, IL6R, SGK, neuropilin, Gabrg1, Ata3, Cebpd, and GADD45a; and (b) determining whether the chemical agent prevents or reverses a change in gene expression, caused by the glucocorticoid receptor agonist, of the member, or the group of members, of the group of genes, thereby determining whether the chemical agent is likely to be an antagonist of glucocorticoid receptor activity.

The representative methods for culturing and contacting neuron-like cells with a chemical agent for measuring gene expression and for comparing levels of gene expression, described in connection with the methods for identifying potential modulators of glucocorticoid receptor activity, are also useful in the practice of the methods for determining whether a chemical agent is an antagonist of glucocorticoid receptor activity.

In another aspect, the present invention provides in vivo methods for identifying potential modulators of glucocorticoid receptor activity. The methods of this aspect of the invention include the steps of (a) administering a chemical agent to a test animal; and (b) measuring the expression of a member, or group of members, of the group of genes consisting of Sult1a1, RGS2, RGS4, IL6R, SGK, neuropilin, Gabrg1, Ata3, Cebpd, and GADD45a in tissue obtained from the test animal administered with the chemical agent; wherein a change in expression of a member, or group of members of the group consisting of Sult1a1, RGS2, RGS4, IL6R, SGK, neuropilin, Gabrg1, Ata3, Cebpd, and GADD45a relative to an animal not administered the chemical agent is indicative of the chemical agent possessing the ability to modulate glucocortocoid receptor activity in vivo.

Animal models can be used to identify potential modulators of glucocorticoid receptor activity in accordance with this aspect of the invention. The chemical agent may be administered to the animal via any suitable route of administration using techniques known to those of skill in the art. For example, the chemical agent may be administered in a pharmaceutical composition. Conventional methods, known to those of ordinary skill in the art of medicine, can be used to administer the pharmaceutical composition to the mammalian subject via conventional routes (e.g., oral, subcutaneous, intrapulmonary, transmucosal, intraperitoneal, intrauterine, sublingual, intrathecal or intramuscular routes) by standard methods. For example, a chemical agent may be combined or admixed with a pharmaceutically acceptable carrier, vehicle or diluent, which may take a wide variety of forms depending on the form of preparation desired for administration. For example, in preparing the compositions in oral dosage form, any of the usual pharmaceutical media may be employed, such as, for example, water, glycols, oils, alcohols, and the like. An exemplary dose of chemical agent is in the range of from about 0.001 to about 200 mg per kg of subject's body weight per day, preferably about 0.05 to 100 mg/kg/day, or about 1 to 35 mg/kg/day. Alternatively, dosages may be administered on a periodic basis, such as once every other day (e.g., from 0.05 to 100 mg/kg every other day). In some embodiments, the chemical agent is administered on a periodic basis over a time period such as once daily over a period of from 1 to 10 days, such as at least 2 days, at least 4 days, or at least 8 days. Tissue samples are then obtained from the test animals and control animals, and gene expression is assessed for the gene or group of genes of interest, as described herein. In some embodiments, tissue samples are obtained from the test animal's central nervous system, such as from the caudate putamen, raphe or hippocampus of the test animal, as described in Example 2.

The following examples merely illustrate the best mode now contemplated for practicing the invention, but should not be construed to limit the invention.

### Example 1

This Example describes the identification of genes that are regulated by glucocorticoid receptor modulators. These genes can be used in the practice of the present invention to identify modulators of glucocorticoid receptor activity.

Cells: CA77 cells are a rat medullary thyroid carcinoma cell line that was first established by Muszynski et al. (“Glucocorticoids Stimulate the Production of Preprocalcitonin-Derived Secretory Peptides by a Rat Medullary Thyroid Carcinoma Cell Line,” J. Biol. Chem. 258(19):11678-11683, October 1983).

Growth of the CA77 Cells: After passage by trypsinization, the cells were maintained in plating media in a humidified air atmosphere (7.5% CO₂) at 37° C. The composition of the plating media (PM) was 500 ml of DMEM (product number 21063, sold by Invitrogen Corporation, 1600 Faraday Avenue, P.O. Box 6482, Carlsbad, Calif. 92008); 500 ml of F-10 (product number 11550, sold by Invitrogen Corporation); 5 ml of sodium pyruvate (product number 11360, sold by Invitrogen Corporation); and 100 ml certified fetal bovine serum (product number 16000, sold by Invitrogen Corporation).

After 24 hours incubation, the plating media was removed and replaced with a serum-free growth media. The composition of the growth media (GM) was 500 ml of DMEM (product number 21063, sold by Invitrogen Corporation); 500 ml of F-10 (product number 11550, sold by Invitrogen Corporation); 5 ml of sodium pyruvate (product number 11360, sold by Invitrogen Corporation); and 10 ml of ITS supplement (Insulin-Transferrin-Selenium supplement; product number 41400, sold by Invitrogen Corporation). The GM was changed every 48-72 hours.

When cultures reached 90-100% confluency, the GM was aspirated, the monolayers washed with D-PBS (product number 14190, sold by Invitrogen Corporation; without Ca⁺⁺ and Mg⁺⁺ ions) and the cells removed with Trypsin-EDTA (product number 25300, sold by Invitrogen Corporation). The cells were then plated at a 1:3 or 1:4 split ratio in PM and then once again changed to GM after 24 hours. These cultures reached confluency after 7-9 days. Cells were maintained in continuous culture for up to 16 passages and then replaced with cells from cryopreserved stocks.

Assay: CA77 cells were plated into 96 well tissue culture dishes at a density of 8×10⁴ cells per well in 200 μl of PM and the cultures maintained in a humidified air atmosphere (7.5% CO₂) at 37° C. This media was aspirated after 24 hours and replaced with 200 μl of GM and the cultures incubated as before, for an additional 48 hours, at which time the cultures were at approximately 90% confluency.

For the evaluation of agonist activity, the GM was aspirated, replaced with 200 μl of fresh GM containing concentrations of a drug ranging from 0.1 nM to 10,000 nM in a 10-fold dilution series. Control wells of 0 nM drug in GM and 0.1% DMSO in GM were included in the series for each drug. Each drug concentration and the two controls were evaluated on 4 wells. The cultures were maintained in a humidified air atmosphere (7.5% CO₂) at 37° C. for 24 hours.

For the evaluation of antagonist activity, the GM was aspirated, replaced with 200 μl of fresh GM containing 250 nM corticosterone (this is the EC50 of corticosterone) as well as concentrations of the drugs ranging from 0.1 nM to 10,000 nM in a 10-fold dilution series. Control wells of 0 nM drug in GM containing 250 nM corticosterone and 0.1% DMSO in GM (without corticosterone) were included in the series for each drug. Each drug concentration and the two controls were evaluated on 4 wells. The cultures were maintained in a humidified air atmosphere (7.5% CO₂) at 37° C. for 24 hours.

Lysis of Cells and Isolation of mRNA: Two methods were employed for the lysis of the cells and the isolation of mRNA at the end of the 24-hour incubation with the drugs.

One method was the mRNA Catcher™ Method (using Kit number 37001, sold by Invitrogen Corporation). The GM was aspirated from the cells, the cell monolayer was washed once with PBS (product number 10010, sold by Invitrogen Corporation), and then the cells were lysed in 30 μl of the kit lysis buffer containing 1% β-ME for 5 minutes at room temperature. The lysates were snap frozen in the 96-well plate by placing the plate on dry ice pellets, and the plate was then stored at −80° C.

The lysates were thawed at room temperature and then transferred into individual wells in the mRNA Catcher™ plate. The plate was covered and incubated for 90 minutes at room temperature for RNA hybridization.

The lysates were aspirated from the wells without scraping the well sides. 150 μl of wash solution (included in the kit) was added to each well and then aspirated. A total of three washes were carried out. cDNA synthesis was performed directly in the mRNA Catcher™ plate without any elution step.

Another method that was used was the GeneCatche® method (kit sold by Invitrogen Corporation, product number 10010). The GM was aspirated from the cells, the cell monolayer was washed once with PBS and then the cells lysed in 85 μl of the kit lysis buffer containing 5 mM DTT for 5 minutes at room temperature. The lysates were snap frozen in the 96-well plate by placing the plate on dry ice pellets, and the plate was then stored at −80° C.

The lysates were thawed at room temperature and then transferred to individual wells in the GeneCatcher plate. The plate was covered with aluminum foil and incubated at room temperature for 90 minutes for RNA hybridization.

The lysates were removed by quickly flipping over the plate. 100 μl of wash buffer (included in the kit) was added to each well and after one minute of incubation, removed by quickly flipping over the plate. A total of 3 washes were carried out. After the final wash, any remaining liquid was removed from the wells by aspiration. cDNA synthesis was performed directly in the GeneCatcher plate without any elution of mRNA.

Reverse Transcription: 50 μl of a reverse transcription reaction mix (DEPC treated water, RT buffer, MgCl₂, dNTP mix, random hexamers, RNase inhibitor, and MULTISCRIBE RT) was added to each well and incubated at 25° C. for 10 minutes, 48° C. for 30 minutes, and 95° C. for 5 minutes. The reaction was halted by the addition of EDTA. The samples were transferred to a storage plate and stored at −20° C.

CA77 Assay Primer/Probe Sets: The regulation of four genes was studied using the CA77 cell assay. These genes were Sulfotransferase 1a1 (Sult1a1), Regulator of G-protein signaling (RGS2), Interleukin 6 Receptor (IL6R) and Serum/Glucocorticoid regulated kinase (Sgk). Primer and probe sets were generated for use in detecting expression of these genes by real-time RT-PCR.

The forward primers for these genes were rSult1a1-407F (5′-CCCTCAGAGTCTGCTGGATCA-3′) (SEQ ID NO:21), rRGS2-504F (5′-AGGCTACAAGTGGCTGCTTCA-3′) (SEQ ID NO:22), rIL6R-944F (5′-CCTTGCGAGGAGTAAAGCATGT-3′) (SEQ ID NO:23), and rSgk-806F (5′-CATCGAGCACAATGGGACAA-3′) (SEQ ID NO:24).

The reverse primers for these genes were rSult1a1-496R (5′-GTAGAAGTTATAATAGGAGACAACCACAT-3′) (SEQ ID NO:25), rRGS2-566R (5′-AAACGAGGATAAGAGTTGTTCTCCAT-3′) (SEQ ID NO:26), rIL6R-1010R (5′-CTCCACTGGCCAATGTCAAA-3′) (SEQ ID NO:27), and rSgk-873R (5′-GGAGAACCTCAGGAGCGAGAT-3′) (SEQ ID NO:28).

The corresponding probes were rSult1a1-457T (5′-FAM-TTTCGGGCAATGTAGATCACCTTGACCTT-TAMRA-3′) (SEQ ID NO:29), rRGS2-552T (5′-FAM-AGGCTGTACACCCTCTTCTGCGCTGTG-TAMRA-5′) (SEQ ID NO:30), rIL6R-989T (5′-FAM-TCCTCCTTCCCTCGGACCTGCA-TAMRA-3′) (SEQ ID NO:31), and rSgk-829T (5′-FAM-CCACCTTCTGTGGCACGCCTGA-TAMRA-3′) (SEQ ID NO:32), wherein FAM is an abbreviation for carboxyfluorescein and TAMRA is an abbreviation for carboxytetramethylrhodamine. The oligonucleotides labeled with FAM or TAMRA are fluorescent resonance energy transfer (FRET) probes that facilitate detection of amplification products using TaqMan technology.

Real-Time RT-PCR Analysis: Real-time RT-PCR analysis for the determination of relative levels of mRNA was performed as follows. 1 μl of cDNA was added to each well of a 96-well plate with 24 μl of TAQMAN reaction mix (1× Universal Master Mix (ABI); 200 nM forward and reverse rodent GAPDH control primers with the 100 nM control probe, 300 nM forward and reverse gene primers with 200 nM probe. Samples were run on an ABI PRISM 7700 Sequence Detection Instrument (Applied Biosystems) and data collected was analyzed using Merck Biometrics TAQMANPLUS program. Data were plotted using GraphPad Prism (available from GraphPad Software, Inc., 11452 El Camino Real, #215, San Diego, Calif. 92130 USA), and EC50 values were determined using this program using non-linear regression analysis.

Results: The group of compounds selected cause a wide range of effects on the glucocorticoid receptor, including full agonist activity (prednisolone and dexamethasone) and low partial agonist activity (RU-486 and Compound 6). These compounds were chosen based on their varying degrees of agonist activity as assessed by MDA and A549 transactivation assays (Table I), a COS cell transrepression assay (Table I) and GR affinity as assessed in binding studies (Table II).

TABLE I
Agonist Activities of Select Glucocorticoid Receptor Modulators

| | Transactivation, | | Transactivation, | | Transrepression, COS | | |
|---|---|---|---|---|---|---|---|
| | MDA | | A549 | | | % Emax, | % Emax, |
| | IP, nM | Emax, % | IP, nM | Emax, % | IP, nM | 1 uM | 0.3 μM |
| Dexamethasone | 18 | 125 | 3 | 118 | 0.1 | 100 | 100 |
| M-Prednisolone | 86 | 104 | 20 | 124 | 1 | 100 | 100 |
| Compound 2 | 14 | 50 | 5 | 54 | 2 | 88 | 88 |
| Compound 3 | 151 | 17 | 28 | 115 | 10 | 71 | 71 |
| Compound 4 | 105 | 38 | 14 | 28 | 3 | 88 | 74 |
| Compound 5 | 43 | 35 | 8 | 47 | 3 | 100 | 73 |
| Compound 6* | 5 | 95 | 48 | 19 | 10 | 42 | |
| RU-486* | 1 | 100 | 10 | 90 | 5 | 40 | |

*Partial Agonist. The following abbreviations are used in Table I: MDA, human mammary carcinoma cell line; IP, inflection point; Emax, maximal response for a compound.

TABLE II

| | IC50 values (nM) | | |
|---|---|---|---|
| | GR | MR | PR |
| Dexamethasone | 2.2 | 6.5 | >1000 |
| Prednisolone | 13.8 | 0.98 | >1000 |
| Compound 2 | 3.5 | 473 | 120 |
| Compound 3 | 8.4 | | |
| Compound 4 | 2.8 | | |
| Compound 5 | 5.8 | | |
| Compound 6 | 0.51 | | |
| RU-486 | 3.8 | 4140 | 3.9 |

The following abbreviations are used in Table II: MR, mineralocorticoid receptor; GR, glucocorticoid receptor; and PR, progesterone receptor.

In order to reduce false positives, progesterone, estradiol, and aldosterone served as out-groups for non-specific effects. Samples were collected at both 6 hours and 24 hours after contacting the CA77 cells with a compound. Compound doses were taken from an A549 cell molecular profiling study that showed a gradation of activity on a set of 1091 genes, ranging from full activation of 1087 of these genes with dexamethasone to activation of only 38 of these genes with RU-486 (Table III).

TABLE III
A549 Cell Profiling Experiment Data

| | | | | | Com- | | | | | RU- | |
|---|---|---|---|---|---|---|---|---|---|---|---|
| | | | Dexamethasone | Prednisolone | pound 2 | Compound 3 | Compound 4 | Compound 5 | Compound 6 | 486 | |
| | Categories | Agonist | 100 nM | 100 nM | 1 μm | 1 μm | 1 μm | 1 μm | 1 μm | 1 μm | DMSO |
| Up- | DMSO | 0 | 0 | 0 | 11 | 34 | 39 | 12 | 231 | 287 | 1091 |
| regulated | (<0%) | | | | | | | | | | |
| | Low | 0 | 0 | 0 | 58 | 127 | 205 | 125 | 434 | 449 | 0 |
| | (0-33.3%) | | | | | | | | | | |
| | Middle | 0 | 0 | 1 | 274 | 353 | 511 | 540 | 284 | 254 | 0 |
| | (33.3-66.7%) | | | | | | | | | | |
| | High | 0 | 4 | 150 | 376 | 274 | 232 | 326 | 84 | 63 | 0 |
| | (66.7-90%) | | | | | | | | | | |
| | Full | 1091 | 1087 | 940 | 372 | 303 | 104 | 88 | 58 | 38 | 0 |
| | (≧90%) | | | | | | | | | | |
| Down- | DMSO | 0 | 0 | 0 | 22 | 55 | 57 | 13 | 337 | 454 | 1249 |
| regulated | (<0%) | | | | | | | | | | |
| | Low | 0 | 0 | 0 | 83 | 183 | 292 | 158 | 509 | 418 | 0 |
| | (0-33.3%) | | | | | | | | | | |
| | Middle | 0 | 0 | 0 | 395 | 450 | 620 | 679 | 307 | 266 | 0 |
| | (33.3-66.7%) | | | | | | | | | | |
| | High | 0 | 12 | 109 | 444 | 343 | 221 | 321 | 73 | 70 | 0 |
| | (66.7-90%) | | | | | | | | | | |
| | Full | 1249 | 1237 | 1140 | 305 | 218 | 59 | 78 | 23 | 41 | 0 |
| | (≧90%) | | | | | | | | | | |

The GR agonists gave a strong signature that did not overlap with the controls or outgroups. The signature is weakly present in aldosterone-treated cells, which primarily act on the mineralocorticoid receptor (MR), suggesting that aldosterone may also have weak agonist activity on the GR. Similarly, the antagonist Compound 6 also shows a subset of the signature. Compound 6 is also known to have low partial agonist activity, which may explain this response at 24 hours.

In order to select the genes with the greatest potential to distinguish between classes of GR modulators, an ANOVA was run on the dexamethasone-treated samples and the controls (dexamethasone being the compound that is closest to a full agonist of GR). 485 sequences (significant at the p<0.001 level) were selected and projected onto the entire dataset. It was noted that a subset of the GR agonist signature is present at 6 hours after contacting the CA77 cells with dexamethasone and this set increases in intensity at 24 hours after contacting the CA77 cells with dexamethasone. Secondly, the agonist signature is more specific at 6 hours after contacting the CA77 cells with dexamethasone, whereas aldosterone and Compound 6 showed little or no activity. For these reasons, the 6-hour signature was used to classify samples.

A principal component analysis was run on the 6-hour signature. Principal component 1 (PC1) captured the majority of the variance in this dataset with a percent eigenvalue of 77.9 and completely separated the GR agonists from the controls and outgroups. Compound 6 (an antagonist of the glucocorticoid receptor) is classified with the controls, suggesting that a set of markers derived from this analysis may be able to distinguish complete agonists from partial agonists/antagonists. Ideal markers, in addition to predicting the presence of GR activity, would also predict the degree or strength of GR agonism displayed by a compound. Thus, a marker for which expression is drastically increased in response to all agonists would provide less information than a marker for which the response scales with agonist activity.

The dataset was ordered on the basis of the cosine correlation of each gene with PC1 (highest correlation on the bottom, highest negative correlation on top). Seven candidate markers were selected from the top and bottom of this list and the magnitude in expression level of these genes reflects the size of the overall signature of each of the compounds. Specifically, Sult1a1, RGS2, RGS4, IL6R, SGK, Neuropilin, and Gabrg1 were selected and verified by RT-PCR.

Table IV shows the RT-PCR results.

TABLE IV

Relative Change

| | |
|---|---|
| Profiling | TaqMan |

| Gene | 6 hr | 24 hr | 24 hr |
|---|---|---|---|
| Sult1a1 | 2.1 | 11 | 13-158 |
| | 2.1 | 9.9 | |
| | 2 | 6.9 | |
| RGS2 | 7.7 | 8.9 | 3.5-30 |
| IL6R | 3.67 | 4.1 | 1.8-7 |
| Sgk | 2.98 | 3.7 | 1.6-15 |
| RGS4 | −1.98 | −2.1 | −2.4-(−16) |
| Neuropilin | | −1.2 | −1.8-(−4.3) |
| Gabrg1 | NC | 1.88 | 1.3-1.5 |

In Table IV under the “Profiling” heading, three values are associated with gene Sult1a1. These three values are the values obtained using three different probes corresponding to Sult1a1 on the microarray (the three values are in good agreement). Values in parentheses are negative values. NC is an abbreviation for “no change,” meaning that no change in the expression level of this mRNA was detected at the 6-hour time point.

All markers were confirmed as being regulated by GR agonists. Although the absolute magnitude of the changes detected by RT-PCR was greater than those detected by microarray (e.g., Sult1a1 was up-regulated by dexamethasone by 11-fold as detected by microarray, but was up-regulated by dexamethasone by 158-fold as detected by RT-PCR), the relative levels between compounds was stable, with dexamethasone and prednisolone having the highest levels and L-690 having the lowest. From the seven markers (Sult1a1, RGS2, RGS4, IL6R, SGK, Neuropilin, and gabrgl), four (RGS2, Sult1a1, IL6R, and SGK) were chosen as the marker panel for the prediction of GR activity in CA77 cells. A second experiment confirmed the up-regulation of each of these four markers by a GR agonist and the reversal of this effect when RU-486 (a GR antagonist) is co-administered with dexamethasone (a GR agonist).

Three additional markers (Ata3, Cebpd, and GADD45a) were selected on the basis of their 24-hour expression signature alone (i.e., these three additional markers do not necessarily correlate with PC1 in the 6-hour signature). Compound 2 had the weakest effect of the GR agonists in CA77 cells (regardless of method of marker selection). As was observed in previous experiments with cell lines not having a neuron-like phenotype (e.g., A549 lung adenocarcinoma cells), Compound 2 had the largest effect. Therefore, while different cell types may all express GR, they do not necessarily respond to various agonists identically. This demonstrates the need for a screen employing a neuron-like phenotype when predicting the neuron-like response.

Although the genes (Sult1a1, RGS2, RGS4, IL6R, SGK, Neuropilin, Gabrg1, Ata3, Cebpd, and GADD45a) were chosen for their magnitude and direction of response to each compound rather than their biological significance, a number of connections to psychiatric disorders exist for this gene set. RGS2 (regulator of G-protein signaling 2) decreases Gaq signaling by the 5-HT2A receptor and its mRNA is up-regulated by haloperidol (Taymans et al., “Dopamine Receptor-Mediated Regulation of RGS2 and RGS4 mRNA Differentially Depends on Ascending Dopamine Projections and Time,” Eur. J. Neurosci. 19(8):2249-2260, April 2004). RSG2-Knockout mice have reduced dendritic spine density in the hippocampus (Oliveira-dos-Santos et al., “Regulation of T Cell Activation, Anxiety, and Male Aggression by RGS2,” Proc. Nat'l Acad. Sci. USA 97(22):12272-12277, October 2000), a phenotype seen in mice which have undergone olfactory bulbectomy (OBX), which is an established model for depression. OBX mice show reversal of the depression phenotype after treatment with antidepressants and this reversal correlates with a return of hippocampal dendritic spine number back to control levels (Norrholm, S. D. and C. C. Ouimet, “Altered Dendritic Spine Density in Animal Models of Depression and in Response to Antidepressant Treatment,” Synapse 42(3):151-163, December 2001). RGS2-Knockout mice also show a decreased aggression/increased anxiety phenotype and the RGS2 locus affects anxiety phenotype in outbred mouse lines (Yalcin et al., “Genetic Dissection of a Behavioral Quantitative Trait Locus Shows That RGS2 Modulates Anxiety in Mice, Nat. Genet. 36(11):1197-1202, November 2004).

Similarly RGS4 decreases Gα_{q} signaling by the 5-HT1A and 5-HT2A receptors and shows down-regulation of mRNA levels in response to haloperidol (Taymans et al., “Dopamine Receptor-Mediated Regulation of RGS2 and RGS4 mRNA Differentially Depends on Ascending Dopamine Projections and Time,” Eur. J. Neurosci. 19(8):2249-2260, April 2004). RGS4 has also been linked to schizophrenia in multiple populations (Chowdari, K. V., et al., “Association and Linkage Analyses of RGS4 Polymorphisms in Schizophrenia,” Hum. Mol. Genet. 11(12):1373-1380, June 2002; Erratum in Hum. Mol. Genet. 12(14):1781, July 2003; Williams, N. M., et al., “Support for RGS4 as a Susceptibility Gene for Schizophrenia,” Biol. Psychiatry 55(2):192-195, January 2004; Morris, D. W., et al., “Confirming RGS4 as a Susceptibility Gene for Schizophrenia,” Am. J. Med. Genet. 125B(1):50-53, February 2004), and the schizophrenic brain shows decreased RGS4 mRNA in comparison to controls (Mimics et al., “Disease-Specific Changes in Regulator of G-Protein Signaling 4 (RGS4) Expression in Schizophrenia,” Mol. Psychiatry. 6(3):293-301, May 2001).

Neuropilin is a VEGF receptor that is involved in axon guidance and neurogenesis. Sult1a1 inactivates dopamine by sulfation and is located at 16p12.1-p11.2 near a locus linked to bipolar disorder (Ewald, H., et al., “A Possible Locus for Manic Depressive Illness on Chromosome 16p13,” Psychiatr. Genet. 5(2):71-81, 1995; McInnis, M. G., et al., Genome-Wide Scan and Conditional Analysis in Bipolar Disorder: Evidence for Genomic Interaction in the National Institute of Mental Health Genetics Initiative Bipolar Pedigrees,” Biol. Psychiatry 54(11):1265-1273, December 2003).

All of these genes are activated 6 hours after contacting the CA77 cells with a GR agonist and each appears to be central to the changes induced by the GR agonists because of their high correlation to principal component analysis 1 (PC1). It is possible, therefore, that these genes have real mechanistic relevance to the effects of GR modulators on PSD and bipolar disorder.

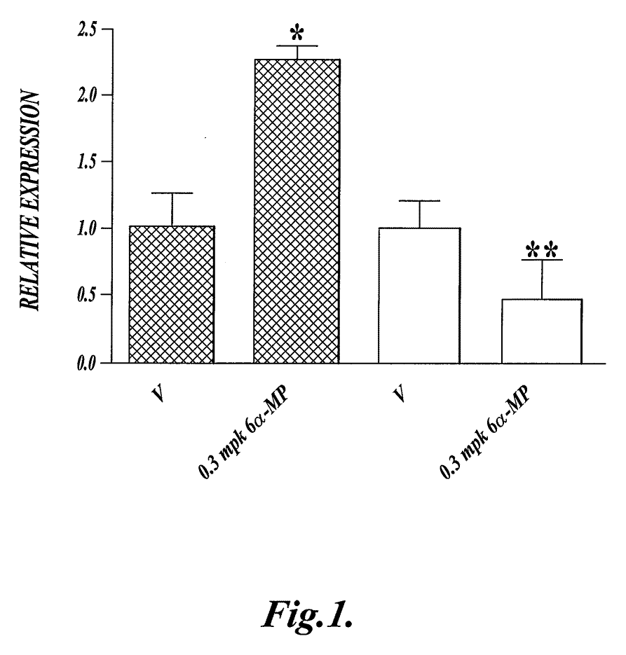

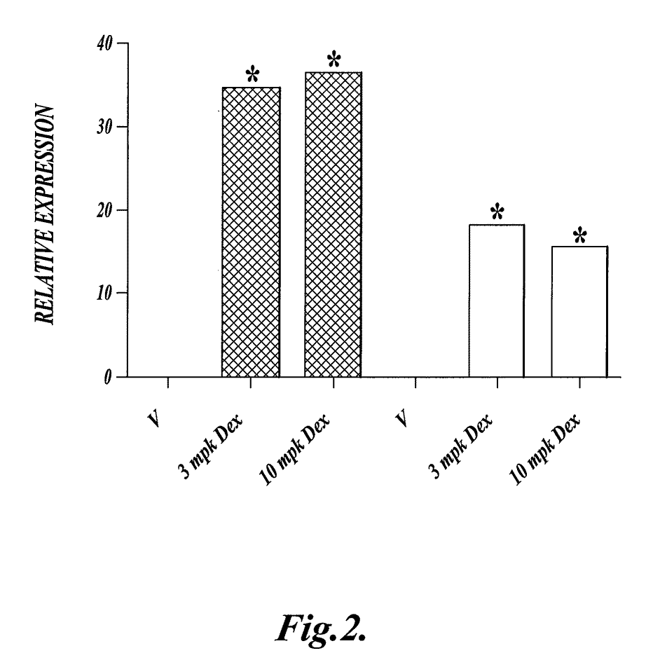

In vivo Studies: To determine if the same genes were regulated by GR agonists in vivo, RT-PCR was performed on the caudate nucleus of rats treated with either 6α-methylprednisolone or dexamethasone. Similar to the results in CA77 cell in vitro and as shown in FIGS. 1 and 2, RGS2 was upregulated 2-fold, RGS4 was down-regulated to about 50% of control levels, and Sult1a1 was drastically upregulated in the caudate nucleus (FIG. 1) and raphe nucleus (FIG. 2). Together, these results strongly suggest that serotinergic and dopaminergic neurotransmission are influenced by agents that modulate GR activity and that these alterations in conjunction with changes in neural architecture (i.e., spine density and neurogenesis) could play a role in the improvement of neurocognitive function observed in bipolar and PMD patients treated with RU-486.

### Example 2

This Example demonstrates that Sult1a1 expression is modulated in vivo in response to agents that modulate GR activity.

Methods: Intact female Sprague Dawley rats (n=7) were dosed via subcutaneous injection once daily for 4 days with the following: vehicle only; dexamethasone (at either 0.03 mg/kg, 0.1 mg/kg, 3 mg/kg or 10 mg/kg); RU-486 at 30 mg/kg; or RU-486 at 30 mg/kg plus dexamethasone (at either 0.03 mg/kg or 0.1 mg/kg).

The rats were sacrificed 6 hours after the final dose and the hippocampus, raphe and caudate nucleus were dissected and placed in RNALate® (Qiagen, Valencia, Calif.) for 24 hours. The tissue was then placed in a fresh tube and stored at −80 C. Total RNA was prepared using Trizol® (InVitrogen, Carlsbad, Calif.) using the manufacturer's standard protocol.

Reverse Transcription:

The total RNA was DNase treated and cDNA was made using the methods described in Example 1 in connection with CA77 cell cDNA preparation.

Sult1a1 Expression Assay:

Relative changes in Sult1a1 gene expression was evaluated using real-time RT-PCR and the Sult1a1 primer/probe set described for the CA77 cell assay studies as described in EXAMPLE 1.

Results:

The results are shown below in TABLE V.

TABLE V

| | Relative Expression | | |
|---|---|---|---|
| | (as compared with vehicle) | | |
| Treatment | Caudate putamen | Raphe | Hippocampus |
| 3 mg/kg | 34.6 +/− 0.1 | 18.25 +/− 0.15 | |
| dexamethasone | (p < 0.001) | (p < 0.001) | |
| 10 mg/kg | 36.6 +/− 0.13 | 15.7 +/− 0.22 | |
| dexamethasone | (p < 0.001) | (p < 0.001) | |
| 0.03 mg/kg | | | 1.78 +/− 0.2 |
| dexamethasone | | | (p < 0.001) |
| 0.1 mg/kg | | | 4.28 +/− 0.11 |
| dexamethasone | | | (p < 0.001) |
| 30 mg/kg | | | 1.18 +/− 0.14 |
| RU-486 | | | |
| 0.03 mg/kg | | | 1.05 +/− 0.17 |
| Dexamethasone | | | |
| 30 mg/kg | | | |
| RU-486 | | | |
| 0.1 mg/kg | | | 2.44 +/− 0.13 |
| Dexamethasone | | | (p < 0.001) |
| 30 mg/kg | | | |
| RU-486 | | | |

The data in Table V shows that treatment with dexamethasone alone resulted in an significant increase in Sult1a1 mRNA in caudate putamen, raphe and hippocampus in test animals as compared to animals receiving vehicle controls. As further shown in TABLE V, Sult1a1 expression in the hippocampus was upregulated in response to dexamethsone (full GR agonist activity), and reversal of this effect was observed when the RU-486 (antagonist activity against GR) was co-administered with dexamethasone. Therefore, these results demonstrate that Sult1a1 expression may be used as a biomarker to screen for agents that are likely to modulate GR activity in vivo.

While illustrative embodiments have been illustrated and described, it will be appreciated that various changes can be made therein without departing from the spirit and scope of the invention.

1. A method for identifying potential modulators of glucocorticoid receptor activity, the method comprising the steps of:
(a) contacting neuron-like cells, in vitro, with a chemical agent;
(b) measuring the expression of a member, or group of members, of the group of genes consisting of Sult1a1, RGS2, RGS4, IL6R, SGK, neuropilin, Gabrg1, Ata3, Cebpd, and GADD45a in the neuron-like cells contacted with the chemical agent; and
(c) determining whether the chemical agent significantly alters the expression of the member, or group of members, of the group of genes consisting of Sult1a1, RGS2, RGS4, IL6R, SGK, neuropilin, Gabrg1, Ata3, Cebpd, and GADD45a, wherein a significant alteration in expression relative to neuron-like cells not contacted with the chemical agent is indicative of the chemical agent possessing the ability to modulate glucocortocoid receptor activity.

2. The method of claim 1, wherein the neuron-like cells are cultured in a liquid medium and the neuron-like cells are contacted with the chemical agent by dissolving the chemical agent in the liquid medium.

3. The method of claim 1, wherein the chemical agent consists essentially of a chemical compound.

4. The method of claim 1, wherein the chemical agent consists essentially of a steroid.

5. The method of claim 1, wherein RT-PCR is used to measure the expression of the member, or group of members, of the group of genes consisting of Sult1a1, RGS2, RGS4, IL6R, SGK, neuropilin, Gabrg1, Ata3, Cebpd, and GADD45a.

6. The method of claim 1, wherein a DNA microarray is used to measure the expression of the member, or group of members, of the group of genes consisting of Sult1a1, RGS2, RGS4, IL6R, SGK, neuropilin, Gabrg1, Ata3, Cebpd, and GADD45a.

7. The method of claim 1, wherein the expression of Sult1a1 is measured.

8. The method of claim 1, wherein the expression of Sult1a1, RGS2, IL6R, and SGK is measured.

9. The method of claim 1, wherein the expression of Sult1a1, RGS2, RGS4, IL6R, SGK, neuropilin, Gabrg1, Ata3, Cebpd, and GADD45a is measured.

10. The method of claim 1, wherein gene expression is measured within 24 hours after contacting the neuron-like cells with the chemical agent.

11. The method of claim 1, wherein gene expression is measured within 6 hours after contacting the neuron-like cells with the chemical agent.

12. The method of claim 1, wherein an increase in expression of the member, or group of members of the group of genes consisting of Sult1a1, RGS2, RGS4, IL6R, SGK, neuropilin, Gabrg1, Ata3, Cebpd, and GADD45a is measured in the neuron-like cells contacted with the chemical agent relative to neuron-like cells not contacted with the chemical agent.

13. The method of claim 1, wherein a decrease in expression of the member, or group of members of the group of genes consisting of Sult1a1, RGS2, RGS4, IL6R, SGK, neuropilin, Gabrg1, Ata3, Cebpd, and GADD45a is measured in the neuron-like cells contacted with the chemical agent relative to neuron-like cell.

14. A method for identifying potential antagonists of glucocorticoid receptor activity, the method comprising the steps of:
(a) comparing the expression of a member, or group of members, of a group of genes in neuron-like cells contacted with a chemical agent and a glucocorticoid receptor agonist, with the expression of the member, or group of members, of the group of genes in neuron-like cells contacted with the glucocorticoid receptor agonist but not with the chemical agent, wherein the group of genes consists of Sult1a1, RGS2, RGS4, IL6R, SGK, neuropilin, Gabrg1, Ata3, Cebpd, and GADD45a; and
(b) determining whether the chemical agent prevents or reverses a change in gene expression, caused by the glucocorticoid receptor agonist, of the member, or the group of members, of the group of genes, thereby determining whether the chemical agent is likely to be an antagonist of glucocorticoid receptor activity.

15. The method of claim 14, wherein the neuron-like cells are cultured in a liquid medium and the neuron-like cells are contacted with the chemical agent by dissolving the chemical agent in the liquid medium.

16. The method of claim 14, wherein the chemical agent consists essentially of a chemical compound.

17. The method of claim 14, wherein the chemical agent consists essentially of a steroid.

18. The method of claim 14, wherein RT-PCR is used to measure the expression of the member, or the group of members, of the group of genes consisting of Sult1a1, RGS2, RGS4, IL6R, SGK, neuropilin, Gabrg1, Ata3, Cebpd, and GADD45a.

19. The method of claim 14, wherein a DNA microarray is used to measure the expression of the member, or the group of members, of the group of genes consisting of Sult1a1, RGS2, RGS4, IL6R, SGK, neuropilin, Gabrg1, Ata3, Cebpd, and GADD45a.

20. The method of claim 14, wherein the expression of Sult1a1 is measured.

21. The method of claim 14, wherein the expression of Sult1a1, RGS2, IL6R, and SGK is measured.

22. The method of claim 14, wherein the expression of Sult1a1, RGS2, RGS4, IL6R, SGK, neuropilin, Gabrg1, Ata3, Cebpd, and GADD45a is measured.

23. The method of claim 14, wherein the glucocorticoid receptor agonist is selected from the group of glucocorticoid receptor agonists consisting of dexamethasone, prednisolone, and corticosterone.

24. The method of claim 14, wherein the expression is measured within 24 hours after contacting the neuron-like cells with the chemical agent.

25. A method for identifying potential modulators of glucocorticoid receptor activity in vivo, the method comprising:
(a) administering a chemical agent to a test animal; and
(b) measuring the expression of a member, or group of members, of the group of genes consisting of Sult1a1, RGS2, RGS4, IL6R, SGK, neuropilin, Gabrg1, Ata3, Cebpd, and GADD45a in tissue obtained from the test animal administered with the chemical agent; wherein a change in expression of a member, or group of members of the group consisting of Sult1a1, RGS2, RGS4, IL6R, SGK, neuropilin, Gabrg1, Ata3, Cebpd, and GADD45a relative to an animal not administered the chemical agent is indicative of the chemical agent possessing the ability to modulate glucocortocoid receptor activity in vivo.

26. The method of claim 25, wherein the expression of Sulta1is measured.

27. The method of claim 25, wherein the expression of Sult1a1, RGS2, and RGS4 is measured.
